(19) United States
(12) Patent Application Publication
(10) Pub. No.: US 2026/0216396 A1
(43) Pub. Date: Jul. 30, 2026

(54) METHODS AND SYSTEMS FOR DISPENSING A VOLATILE MATERIAL

(71) Applicant: S. C. JOHNSON & SON, INC., Racine, WI (US)

(72) Inventors: Sebastian D. HASIK, Antioch, IL (US); Thomas P. GASPER, Germantown, WI (US)

(21) Appl. No.: 19/143,801

(22) PCT Filed: Dec. 20, 2023
(86) PCT No.: PCT/US2023/085092

§ 371 Date: Jun. 26, 2025

Related U.S. Application Data
(60) Provisional application No. 63/435,494, filed on Dec. 27, 2022.

(51) Int. Cl. A61L 9/03; G01K 1/02; G01K 7/02; G01K 7/22; H05B 1/02

(52) U.S. Cl. CPC A61L 9/037; G01K 1/026; G01K 7/02; G01K 7/22; H05B 1/0252; A61L 2209/111; A61L 2209/133; A61L 2209/134

(57) ABSTRACT

An apparatus for dispensing a volatile material includes a power supply, a heating element, a temperature detection device, a refill, and a controller. The controller is in communication with the power supply, the heating element, and the temperature detection device. The heating element is in thermal communication with the refill and the temperature detection device.

### CROSS REFERENCE TO RELATED APPLICATIONS

This application claims benefit of priority to U.S. Provisional Application No. 63/435,494, filed Dec. 27, 2022, the contents of which is incorporated by reference in its entirety.

### REFERENCE REGARDING FEDERALLY SPONSORED RESEARCH OR DEVELOPMENT

Not applicable

### SEQUENTIAL LISTING

Not applicable

### BACKGROUND OF THE DISCLOSURE

### 1. Field of the Disclosure

The present disclosure relates generally to methods and systems for dispensing volatile materials, and more particularly, to systems and methods for volatizing a liquid containing a mixture of fragrances to achieve a consistent volatile material intensity across a variety of use cases to improve a user's experience.

### 2. Description of the Background of the Disclosure

A multitude of volatile material diffusion devices or diffusers exist in the marketplace. Many of such devices are passive devices that require only ambient air flow to disperse the liquid active material therein. Other devices are battery-powered or receive household power via a plug extending from the device. Some known diffusers include a heating element for heating a volatile material to promote vaporization thereof. Other diffusers employ a fan or blower to generate air flow to direct volatile material out of the diffuser into the surrounding environment. Still other diffusers that dispense volatile materials utilize ultrasonic means to dispense the volatile materials therefrom. Fragrance compositions that are used in the aforementioned volatile material dispensers are composed of a mixture of volatile perfume raw materials, and it is not uncommon for a fragrance composition to be composed of over twenty different perfume raw materials. The chemical properties of perfume raw materials in a fragrance composition often vary widely in terms of polarity, density, vapor pressure, flash point, and other properties.

A problem with past volatile material dispensers is that their heating element characteristics and the characteristics of the volatile material may vary when used in different environments, which alters how a volatile material is evaporated and sensed by a user. Thus, a volatile material dispenser located at a first location may dispense the volatile material at a different rate than the volatile material dispenser located at a second location having a different relative climate than the first location. The difference in the rate at which the volatile material is dispensed can result in different concentrations of volatile materials dispensed into the surrounding environment among the volatile material dispensers located at first and second locations. As a result, systems and methods for dispensing a volatile material that more accurately control the volatile material output may be useful.

### SUMMARY OF THE DISCLOSURE

In some embodiments, a volatile material dispenser comprises a power supply, a heating element, a first temperature detection device, a second temperature detection device, a refill, and a controller. The controller is in communication with the power supply, the heating element, the first temperature detection device, and the second temperature detection device. The heating element is in thermal communication with the refill and the second temperature detection device. The first temperature detection device is in thermal communication with the refill. In some embodiments, the first temperature detection device and/or the second temperature detection device is a thermistor. In some embodiments, the first temperature detection device and/or the second temperature detection device is a thermocouple. In some embodiments, the volatile material dispenser comprises an air displacement mechanism. In some embodiments, the air displacement mechanism is an air vent. In some embodiments, the volatile material dispenser comprises a second temperature detection device that is in communication with the controller and a volatile material within the refill. In some embodiments, the volatile material dispenser includes an air flow detection mechanism that is in communication with the controller. In some embodiments, the volatile material dispenser includes a volatile material concentration detector that is in communication with the controller. In some embodiments, the volatile material dispenser is in communication with one or more smart home devices via a communication network.

In some embodiments, a method of emitting a volatile material from a volatile material dispenser includes the steps of determining if an air flow level in the environment is below a predetermined threshold suitable for operation of the volatile material dispenser. The method further includes the steps of operating a duty cycle if the air flow level is below the predetermined threshold or waiting a predetermined amount of time before re-determining if the air flow is below the predetermined threshold suitable for operation of the volatile material dispenser. In some embodiments, the air flow level is measured using an ambient conditions monitor. In some embodiments, the air flow level is determined using data received from an external device that may be a smart home device. In some embodiments, the volatile material dispenser comprises of at least two temperature detection devices.

In some embodiments, a method of emitting a volatile material from a volatile material dispenser having a heating element and a refill containing the volatile material includes the step of determining if the volatile material concentration in an environment is above or below a predetermined volatile material concentration threshold. The method further includes the steps of moving the heating element closer to the refill if the volatile material concentration is below the predetermined volatile material concentration threshold and moving the heating element away from the refill if the volatile material concentration is above the predetermined volatile material concentration threshold. In some embodiments, the volatile material concentration is measured using a volatile material detector. In some embodiments, the volatile material concentration is determined using by using data received from an external device that may be a smart home device. In some embodiments, the volatile material dispenser comprises at least two temperature detection devices.

In some embodiments, a method of emitting a volatile material from a volatile material dispenser having a heating element and a refill containing the volatile material includes the step of determining the temperature of the heating element and the temperature of the volatile material within the refill. The method further includes the steps of using the temperature of the heating element and the volatile material within the refill to calculate an energy transfer rate between the heating element and the volatile material within the refill needed to achieve a particular volatile material evaporation rate. The method further includes adjusting the proximity of the heating element and the refill containing the volatile material to achieve the energy transfer rate for the particular volatile material evaporation rate. In some embodiments, the temperature of the heating element and/or the volatile material within the refill is determined using a thermocouple or a thermistor.

In some embodiments, a method of emitting a volatile material from a volatile material dispenser having a heating element and a refill containing the volatile material includes the step determining if an air flow level in the environment is above a predetermined threshold suitable for operation of the volatile material dispenser. The method further includes the steps of operating a duty cycle if the air flow level is above the predetermined threshold or waiting a predetermined amount of time before re-determining if the air flow is above the predetermined threshold suitable for operation of the volatile material dispenser. In some embodiments, the air flow level is measured using an ambient conditions monitor. In some embodiments, the air flow level is determined using data received from an external device that may be a smart home device. In some embodiments, the volatile material dispenser comprises at least two temperature detection devices. In some embodiments, the volatile material dispenser may comprise an air displacement mechanism that is an air vent.

In some embodiments, a volatile material dispenser may comprise a power supply, a heating element, a temperature detection device, a refill, and a controller. The controller may be in communication with the power supply, the heating element, and the temperature detection device. The temperature detection device may be configured to be in thermal communication with the refill. The heating element may be in thermal communication with the refill. In some embodiments, the refill comprises a cartridge, a volatile material, and a wick. In some embodiments, the temperature detection device is in thermal communication with the wick. In some embodiments, the temperature detection device is a thermistor and/or a thermocouple. In some embodiments, the volatile material dispenser also comprises an air displacement mechanism. The air displacement mechanism may be an air vent. In some embodiments, the volatile material dispenser also comprises an air flow detection mechanism that is in communication with the controller. In some embodiments, the volatile material dispenser further comprises a volatile material concentration detector that is in communication with the controller. In some embodiments, the volatile material dispenser is in communication with one or more smart home devices via a communication network.

In some embodiments, a volatile material dispenser comprises a housing and a first controller that is in electrical communication with a heating element that is in thermal communication with a refill and electrical communication with a first power supply. Further, the volatile material dispenser may comprise a base that comprises a second controller that is in communication with an air displacement mechanism and a second power supply. In some embodiments, the housing and the base can be configured to be removably coupled with each other. In some embodiments, the volatile material dispenser has at least one operational cycle when the base is not coupled to the housing. In some embodiments, the volatile material dispenser has at least one operational cycle when the base is coupled to the housing that is different than at least one operational cycle when the base is not coupled to the housing. In some embodiments, the volatile material dispenser has at least one communication system that is connected to at least one controller. In some embodiments, the volatile material dispenser has a first communication system and a second communication system such that the first communication system is connected to the first controller, and the second communication system is connected to the second controller. In some embodiments, the housing and the base are coupled using magnets. In some embodiments, the refill further comprises a wick and a container containing a volatile material within.

### BRIEF DESCRIPTION OF THE DRAWINGS

FIG. 1 is a schematic representation of a configuration of a volatile material dispenser as disclosed herein;

FIG. 2 is a first graph illustrating varying volatile material evaporation rates of a volatile material and volatile material dispenser in a first environment and in a second environment;

FIG. 3 is a second graph illustrating varying volatile material evaporation rates of three different volatile materials with different embedded fragrances based on a power output or temperature of a heater of the volatile material dispensers disclosed herein;

FIG. 4 is a schematic representation of another configuration of a volatile material dispenser;

FIG. 5 is a schematic representation of yet another configuration of a volatile material dispenser;

FIG. 6 is a schematic representation of still another configuration of a volatile material dispenser;

FIG. 7 is a schematic representation of another configuration of a volatile material dispenser;

FIG. 8 is a schematic representation of another configuration of a volatile material dispenser;

FIG. 9 is a flow chart of a method of operating a volatile material dispenser;

FIG. 10 is a flow chart of another method of operating a volatile material dispenser;

FIG. 11 is a flow chart of yet another method of operating a volatile material dispenser;

FIG. 12 is a flow chart of still another method of operating a volatile material dispenser;

FIG. 13 is a flow chart of yet another method of operating a volatile material dispenser;

FIG. 14 is a flow chart of another method of operation a volatile material dispenser;

FIG. 15 is a schematic representation of another configuration of a volatile material dispenser; and

FIG. 16 is a schematic representation of yet another configuration of a volatile material dispenser.

### DETAILED DESCRIPTION OF THE DRAWINGS

The present disclosure relates to volatile material dispensing systems and methods that provide for controlling a rate of energy transfer of a heating element to a volatile material to offer a more consistent volatile material concentration and fragrance scent across varying environments to enhance a user's experience. The differences in characteristics of the function of the volatile material dispenser and the volatile material may be attributed to differences in location characteristics such as humidity, elevation, air pressure, temperature and/or other environmental factors. The differences in environmental factors can influence the evaporation characteristics of the volatile material and the efficacy of the heating element. Thus, due to variances in location characteristics, volatile materials dispensed by volatile material dispensers may achieve concentrations and fragrance characteristics that are undesirable for a user. Thus, a variety of different control techniques are used to dictate the energy transfer rate from the heater. By regulating the energy applied to a volatile material, the volatile material concentration and fragrance characteristics from the volatile material in a first environment can be controlled and adjusted to be consistent across a plurality of environments, which can improve a user's experience. The systems and methods disclosed herein allow the volatile material dispensing system to control an energy transfer rate of the heating element to the volatile material, and to thus control a volatile material concentration and fragrance experience in an environment to achieve consistent volatile material concentrations and fragrance experiences across a plurality of different environments.

By controlling the energy transfer rate of the heating element in a targeted fashion, the volatile material dispenser can adjust or modify the volatile material evaporation rate and fragrance characteristics depending upon environmental factors and location characteristics. Thus, the volatile material dispenser may manipulate the volatile material evaporation rate and evaporation process in a way that reduces differentiation in volatile material concentrations and fragrance characteristics between two different surrounding environments. To that end, the systems and methods disclosed herein address the inconsistencies of volatile material concentrations and the inconsistencies in fragrance experiences that are the result of the volatile material dispensers performing differently in varying environments. Throughout the disclosure, the terms “about” and “approximately” refer to a range of values ±5% of the numeric value that each term precedes. As noted herein, all ranges disclosed within this application are inclusive of the outer bounds of the range.

The term “fragrance,” as used herein, refers to any substance or a mixture of substances such as a perfume designed to emit an aromatic scent. A wide variety of chemicals are known for fragrance (i.e., perfume) uses, including materials such as aldehydes, ketones, and esters. More commonly, naturally occurring plant and animal oils and exudates comprising complex mixtures of various chemical components are known for use as fragrances. The present disclosure relates to volatile material dispensers that are configured to emit any fragrance or combinations of fragrances. Many fragrances comprise a number of different perfume raw materials, each having their own chemical characteristics or properties, which generally vary in terms of polarity, density, vapor pressure, flash point, and other properties.

The fragrances and other volatile compositions of the present application may comprise a single chemical or may comprise a sophisticated complex mixture of natural and synthetic chemical components, all chosen to provide any desired odor or effect. For example, the fragrances and perfumes of the present application may comprise one or more perfume raw materials. The term “perfume raw materials,” as used herein, refers to any compound (e.g., those having molecular weight of at least 100 g/mol) or substance that are useful in imparting an odor, fragrance, essence, or scent either alone or in combination with other “perfume raw materials.” Mixtures of perfume raw materials are known by those skilled in the art of fragrances and perfumes as “accords.” The term “accord,” as used herein, refers to a mixture of two or more perfume raw materials which are artfully combined to impart a scent, odor, essence, or fragrance characteristic.

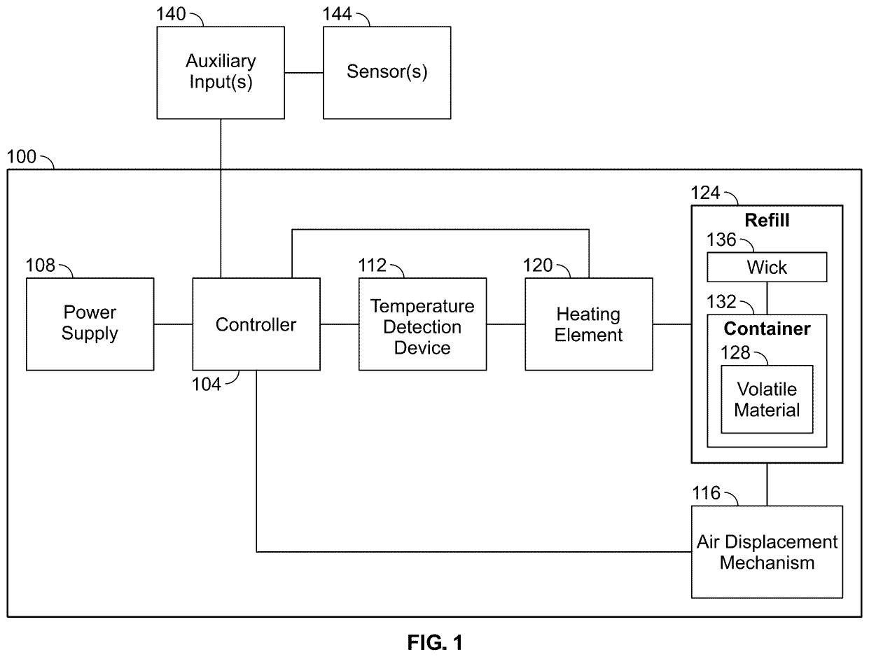

Referring to FIG. 1, a schematic representation of a volatile material dispenser 100 is shown. The volatile material dispenser 100 comprises a controller 104 that is connected to a power supply 108, a temperature detection device 112, an air displacement mechanism 116, a heating element 120, and a refill 124. The refill 124 includes a volatile material 128 disposed within a container 132. The refill 124 also comprises a fluid delivery system, shown as a wick 136, that is in fluid communication with the volatile material 128 and thermal communication with the heating element 120. The temperature detection device 112 is in thermal communication with the heating element 120 and in electrical communication with the controller 104. The heating element 120 is in thermal communication with the volatile material 128 through the wick 136. The volatile material 128 is also in fluid communication with the air displacement mechanism 116 via the wick 136. The temperature detection device 112 may be configured to be in direct contact with the heating element 120, or the temperature detection device 112 may be in thermal communication with the heating element 120 such that the temperature detection device 112 can measure a small change in temperature, e.g., at least a change of 0.1° C., or the change of another parameter. In some embodiments, the temperature detection device 112 is an integral component of the heating element 120. The temperature detection device 112 and the controller 104 are in electrical communication using a wired connection or a wireless connection. Additionally, the controller 104 is in electrical communication with the air displacement mechanism 116.

Referring to FIG. 1, the controller 104 is used to control operation of the heating element 120 and other electrical components such as the air displacement mechanism 116. The controller 104 may include a voltage regulator and may also include a number of other electrical components, including capacitors, resistors, inductors, fuses, diodes, and so forth. In some embodiments, the controller 104 may include a timer, which may allow the controller 104 to shut off specific electronic components for a predetermined amount of time after a predetermined amount of time of use.

Still referring to FIG. 1, the controller 104 may also include a plurality of components disposed thereon, which may include a wireless communication device or receiver that may be a module that supports wireless communication. The controller 104 may further include a first regulator and a second regulator, either of which may be a voltage regulator. The wireless communication system may support Bluetooth® Low Energy (BLE) wireless communication, Wi-Fi, or other types of wireless communication. In a preferred embodiment, the wireless communication system includes one or more of onboard crystal oscillators, chip antenna, and/or passive components. The wireless communication system may support a number of peripheral functions, e.g., application deliver controller (“ADC”), timers, counters, pulse width modulation (“PWM”), and serial communication protocols. Some of the serial communication protocols used through the wireless communication system's programmable architecture may include I2C, universal asynchronous receiver/transmitter (“UART”), and serial peripheral interface (“SPI”). The controller 104 may be or include a microcontroller unit (“MCU”) and/or an application-specific integrated circuit (“ASIC”). The controller 104 may include a processor, a flash memory, and additional components not specifically noted herein. The controller 104 may utilize one or more auxiliary inputs 140 that are configured to provide information such as user preferences, switch selection, and environmental factors.

Still referring to FIG. 1, the power supply 108 may be configured to receive a USB-C type plug, that can be used to charge or power the volatile material dispenser 100. In some embodiments, the volatile material dispenser 100 may receive power from a wall outlet, a car lighter socket, or another source of power as the power supply 108. In some embodiments the power supply 108 may be a battery which could include a rechargeable battery, a one-time use battery, a lead-acid battery, a nickel-cadmium battery, a nickel-metal hybrid battery, a lithium-ion battery, an alkaline battery, a zinc-carbon battery, a coin cell battery, a zinc-air battery, a sealed lead-acid battery, or any other device known in the art that holds energy in the form of chemicals. In some embodiments, the power supply 108 may use a combination of battery types and/or the power supply 108 may be a combination of power sources such as a battery and a wall charger. In some embodiments, the power supply 108 may implement power conditioning to transform the line voltage to 5V DC. In some embodiments, the power supply 108 may not implement power conditioning.

In some embodiments, the volatile material dispenser 100 may comprise a base and a housing that are detachable from one another. The base may include the air displacement mechanism 116 and a charging apparatus for the power supply 108, such as a battery. The housing may contain the refill 124, the heating element 120, the controller 104, and the power supply 108. In these embodiments, the volatile material dispenser 100 may be configured to be charged on the base that is connected to a wall outlet or other power source. While the volatile material dispenser 100 is connected to the base, the volatile material dispenser 100 may be configured to use the air displacement mechanism 116 to disperse the evaporated volatile material 128 into the surrounding environment. When the housing of the volatile material dispenser 100 is not attached to the base, the volatile material dispenser 100 may be configured to be placed in a more desirable location that is beyond the reach of a power source. Thus, the volatile material dispenser 100 may operate without the air displacement mechanism 116 by taking advantage of a placement that is closer in proximity to the user or another desirable location.

In some embodiments, the temperature detection device 112 is in electrical communication with the controller 104 and in thermal communication with the heating element 120. In some embodiments, the temperature detection device 112 is a negative temperature coefficient (“NTC”) thermistor. In some embodiments, the temperature detection device 112 is a positive temperature coefficient (“PTC”) thermistor. In some embodiments, the temperature detection device 112 is a thermocouple or any similar temperature measuring mechanisms.

Still referring to FIG. 1, the heating element 120 is in thermal communication with the volatile material 128 and the temperature detection device 112. In some embodiments, the heating element 120 may be a ceramic annular disk with an inlayed potted metal oxide resistor. In some embodiments, the heating element 120 comprises ceramic with an inlayed potted metal oxide resistor that is not in an annular shape. Further, the heating element 120 may be a tubular metal oxide resistor, a kapton heater, a foil heater, or a copper heating coil. In some embodiments, the heating element 120 comprises a honeycomb configuration such that it is considered a honeycomb heater. Further, the heating element 120 may be configured to receive the wick 136 that extends upwardly into contact with the heating element 120. Alternatively, the heating element 120 may be a contactless laser LED or a consumable heating element such as a tungsten filament. In some embodiments, the heating element 120 may be integrated in the refill 124. In some embodiments, the heating element 120 comprises a plurality of heating elements that surround the wick 136. The plurality of heating elements may be arranged in such a way that they are able to sequentially heat the wick 136. In some embodiments, the heating element 120 may be a nichrome wire that is embedded in the wick 136 that is attached to and/or along portions of the container 132 of the refill 124. In some embodiments, the heating element 120 may be a pin-point heater. In some embodiments, the heating element 120 may comprise one or more resistors.

Still referring to FIG. 1, the volatile material 128 may be in direct contact with the heating element 120 through the wick 136. In some embodiments the wick 136 is not in direct contact with the heating element 120. In some embodiments, the refill 124 is in thermal communication with the heating element 120 such that the wick 136 brings the volatile material 128 close enough in proximity to heating element 120 to facilitate evaporation of the volatile material 128 faster than an evaporation in a controlled setting. In some embodiments, the heating element 120 is in communication with the refill 124 via the wick 136 and transports the volatile material 128 from the container 132 to the heating element 120. In some embodiments, the wick 136 may be a sintered wick such as a POREX® Wick. In some embodiments, the fluid delivery system shown as the wick 136 in FIG. 1 is another different type of liquid transfer mechanism. In some embodiments, the fluid delivery system may be a gravity fed injector or an apparatus for storing fluid that has a connection to a drip pipe disposed below the apparatus. In some embodiments, the fluid delivery system is a solenoid or an electronic fuel injector type dispenser that can periodically deliver a metered amount of volatile material from the refill 124 to the heating element 120 for volatilization. The timing and delivery of volatile material 128 can be controlled by the controller 104, which can then provide a precise heating sequence to the heating element 120 based upon when volatile material 128 is placed into contact with the heating element 120. Regardless of the type of fluid delivery system (e.g., the wick 136), the heating element 120 is in thermal communication (e.g., direct contact or indirect contact) with liquid carried by the fluid delivery system. In some embodiments, the wick 136 is a sintered wick to which a spring is coupled to facilitate contact between the wick 136 and the heating element 120. The sintered wick with the spring may offer compliant contact to accommodate varying tolerances of components of the volatile material dispenser 100. In some embodiments, the heating element 120 and the refill 124 may be in communication by the heating element 120 extending into a small cylinder cutout within the top of the refill 124.

Still referring to FIG. 1, the refill 124 may be a pre-dosed pad or gel. In some embodiments, the refill may be integral and nonremovable from the volatile material dispenser 100. In some embodiments, the refill 124 may be a refillable container that may include a cartridge. The cartridge may be configured to include additional elements, such as a base that is coupled with the cartridge. In some embodiments, the refill 124 may be a glassomizer, a clearomizer, or a catromizer, or may be another type of device that is used to deliver liquid to vapor. In some embodiments, the refill 124 includes a tank defining the container 132. The refill 124 may be configured to be removeable from the volatile material dispenser 100. In some embodiments, the refill 124 is configured for one-time use such that the wick 136 and the heating element 120 are embedded within the refill 124. In some embodiments, the refill 124 may be configured to be used for multiple uses and may also be detachable from the volatile material dispenser 100. In some embodiments, the refill 124 may be an Aspire™ Nautilus tank that implements an adjustable airflow ring at a bottom end thereof that allows for various heat settings. In some embodiments, the refill 124 includes a removable tank, which may comprise Pyrex®.

In some embodiments, the refill 124 holds the volatile material 128 that may include one or more compositions, which may be any suitable liquid or liquids, and may include one or more active ingredients. Active ingredients include, but are not limited to, one or more of a cleaner, an insecticide, an insect repellent, an insect attractant, a disinfectant, a mold or mildew inhibitor, an antimicrobial, a fragrance comprised of one or more aroma chemicals, a disinfectant, an air purifier, an aromatherapy scent, an antiseptic, an odor eliminator, a positive fragrancing active material, an air-freshener, a deodorizer, a medicinal component, an inhalant (e.g., for relieving a cough or congestion), or the like, and combinations thereof. The volatile material dispenser 100 disclosed herein may be used as a pest control product that has the ability to operate in both an unfragranced and a fragranced repellent mode (e.g., at a lower temperature setting which outputs a very light or negligible fragrance and at a higher temperature setting which outputs a fragrance).

Still referring to FIG. 1, the air displacement mechanism 116 is in electrical communication with the controller 104 and in fluid communication with the volatile material 128. The air displacement mechanism 116 may be a fan at the base of the volatile material dispenser 100 that blows the evaporated volatile material from the refill 124 through and out of the volatile material dispenser 100. In some embodiments, the air displacement mechanism 116 may be a secondary heating element (not shown) that creates a convection tube within the volatile material dispenser 100 that carries the evaporated volatile material from the refill 124 away from the volatile material dispenser 100. In some embodiments, the air displacement mechanism 116 may include internal or external structure that is configured to take advantage of naturally occurring diffusion or the surrounding air flow in the environment to provide enhanced air flow and/or mitigate power consumption of the volatile material dispenser 100. In some embodiments, the air displacement mechanism 116 is an air pump or a pneumatic pump. Alternatively, the air displacement mechanism 116 may be a venturi tube with a secondary heating element (not shown) separate from the first heating element 120. The venturi tube may be configured to create a pressure differential that may promote air movement and function as the air displacement mechanism 116. In some embodiments, the air displacement mechanism 116 is a ceiling fan wherein the volatile material dispenser 100 is mounted on one of the fans blades. In some embodiments, the air displacement mechanism 116 may be a diaphragm, a vacuum pump, or a compressor. In some embodiments, the air displacement mechanism 116 is an external apparatus that moves and has the volatile material dispenser 100 attached to the external apparatus. The external apparatus that moves may be a car, a bike, a scooter, a dog, a treadmill, a stationary bike, a door, a window, or any other object that moves.

Still referring to FIG. 1, the air displacement mechanism 116 may be a vehicle air vent and the volatile material dispenser 100 may be configured to be mounted to the vehicle air vent. In some embodiments, the air displacement mechanism 116 may be configured to have an adjustable nozzle that is designed to influence the exit of the evaporated volatile material into the surrounding environment. In such embodiments, the nozzle may be configured to increase or decrease the exit angle to influence the dispersion of the evaporated volatile material into the surrounding environment. In some embodiments, the air displacement mechanism 116 uses ionic wave generation.

In some embodiments, the volatile material dispenser 100 may not include the air displacement mechanism 116. Thus, the evaporated volatile material from the refill 124 may be a passive dispenser and is configured to disperse amongst the environment through ordinary diffusion and/or other environmental forces. In some embodiments, the air displacement mechanism 116 may be configured to push air through a hose which enters an emission cavity within the refill 124 and mixes with the evaporated volatized material within the tank of the refill 124 after which the air and volatile combination is expelled out of a nozzle of the refill 124 due to a pressure differential caused by the air displacement mechanism 116.

Still referring to FIG. 1, the volatile material dispenser 100 may be configured to control the volatile material concentration in the environment by controlling an amount of the volatile material 128 that is evaporated from the refill 124 by using the temperature detection device 112 to monitor the power output of the heating element 120. In some embodiments, the controller 104 may be configured to monitor the temperature of heating element 120 using the temperature detection device 112. In some embodiments, the temperature detection device 112 may be a PTC thermistor. In such embodiments, the PTC thermistor may be in contact or close proximity to the heating element 120 such that the PTC thermistor has a resistance value that reacts to a heat output of the heating element 120. The controller 104 is connected to the PTC thermistor and may be configured to detect the heat output and/or temperature of the heating element 120 by determining a change in resistance of the PTC thermistor. In some embodiments, the temperature detection device 112 may be a NTC thermistor. In such embodiments, the NTC thermistor may be in contact or close proximity to the heating element 120 such that the NTC thermistor has a resistance value that reacts to a heat output of the heating element 120. The controller 104 is connected to the NTC thermistor and may be configured to detect a heat output and/or temperature of the heating element 120 by using the change in resistance of the NTC thermistor. In some embodiments, the temperature detection device 112 may be a thermocouple. In such embodiments, the thermocouple is in contact or close proximity to the heating element 120 such that the thermocouple has a voltage value that reacts to a heat output of the heating element 120. The controller 104 is connected to the thermocouple and may be configured to detect a heat output and/or temperature of the heating element 120 by using the change in voltage of the thermocouple.

Still referring to FIG. 1, the volatile material 128 within the refill 124 is in fluid communication with the heating element 120, which may be a pin-point heater. By using a heater such as a pinpoint heater, desirable temperatures can be achieved relatively quickly. In some embodiments, the heating element 120 may be turned “on” for a few seconds, every few minutes to achieve an activation period. During the activation period, the power level delivered to the heating element 120 may be varied (e.g., between a range of power settings) to achieve an average target power setting (e.g., 5.0 W) over the activation period. In other examples, the heating element 120 may be activated and held at a constant power level throughout the activation. Through the use of PWM, the heating element 120 can be adjusted to a range of different temperatures, and by achieving varying temperatures, different volatile material evaporation rates can be produced and output.

Since small deviations in temperature may cause different volatile material concentrations or different scent notes in the environment that are perceptible by a user, some embodiments of the present disclosure may implement the auxiliary inputs 140 that may include discrete user inputs through settings that provide for different intensities that are perceptible by a user, e.g., “low,” “medium,” and “high.” In some embodiments, 4.0 Watts (W) of power may be associated with a “low” setting, 5.0 W of power may be associated with a “medium” setting, and 6.0 W of power may be associated with a “high” setting. In some embodiments, the user may be able to input a plurality of fragrance preferences as one of the auxiliary inputs 140. The volatile material dispenser 100 may be configured to utilize the plurality of fragrance preferences to oscillate between different run cycles that correlate with a preferred fragrance characteristic of the evaporated volatile material 128. In some embodiments, the user may input a selection of “lavender,” “jasmine,” and “vanilla.” The volatile material dispenser 100 may be configured to cycle through a low heat cycle to produce a “lavender” fragrance followed by a medium heat cycle to produce a “jasmine” fragrance and then a high heat cycle to produce a “vanilla” fragrance. In some embodiments, the controller 104 may have a randomizer that determines what intensity of heat to use for a given cycle. Thus, the fragrance output from the evaporated volatile material may be varied to prevent the user from experiencing fragrance habituation. This is discussed in more detail below.

Still referring to FIG. 1, the volatile material dispenser 100 may be configured to move the heating element 120 closer to or farther from the volatile material 128 depending on an intensity selection of the user and/or other factors including environmental factors and measurements from the temperature detection device 112. In such embodiments, the volatile material dispenser 100 may be configured to move the heating element 120 closer to the refill 124 to increase the intensity of the heat transferred from the heating element 120 to the refill 124. Thus, the amount of the volatile material 128 evaporated into the environment may increase as a result of an increased rate in energy transfer from the heating element 120 to the volatile material contained within the refill 124. In contrast, the volatile material dispenser 100 may be configured to move the heating element 120 away from the refill 124 to decrease the amount and intensity of the energy transferred from the heating element 120 to the refill 124. Thus, the amount and intensity of the volatile material released into the environment may decrease.

In some embodiments, the volatile material dispenser 100 may be configured to control the rate of volatile material evaporation by moving the heating element 120 along the refill 124, which may comprise a pre-dosed pad or gel, to continuously expose the heating element 120 to a new area or region of the volatile material composition. In some embodiments, the heating element 120 may be moved using a pendulum or another type of oscillating device.

Still referring to FIG. 1, the flow rate of the volatile material 128 from the refill 124 to the heating element 120 through the wick 136 may be controlled to control the amount of the volatile material 128 that is evaporated and released into the environment. In some embodiments, the flow rate of volatile material 128 from the refill 124 to the heating element 120 can be controlled using a secondary heating element (not shown) to heat or cool the refill 124. Heating the refill 124 with the secondary heating element (not shown) may decrease the viscosity of the volatile material 128, thereby increasing the flow from the refill 124 to the heating element 120 via the wick 136. In a similar fashion, the secondary heating element (not shown) may be turned off or the power output may be lowered to increase the viscosity of the volatile material 128 in the refill 124 to reduce the flow rate of the volatile material 128 from the refill 124 to the heating element 120 via the wick 136.

In some embodiments, the volatile material dispenser 100 may use duty cycles to achieve the desired volatile material concentration in the surrounding environment. The volatile material dispenser 100 may be configured to have a power switch that is used to power “on” and “off” the volatile material dispenser 100. When the power switch is activated to an “on” position, the controller 104 can direct electric current to flow to the heating element 120 and the air displacement mechanism 116 using power supplied by the power supply 108. Once a target temperature is achieved, the controller 104 can shut off the electrical current supplied to the heating element 120 while continuously monitoring a current temperature of the heating element 120 using signals provided by the temperature detection device 112 or another environmental sensor arranged on or in proximity to the heating element 120. When the measured temperature drops to a predetermined value, the controller 104 may be configured to restore electrical current to initiate another operation cycle. In some embodiments, a PWM algorithm may be used to allow the heating element 120 to heat up quickly, which in turn may allow a faster fragrance or volatile material release.

Still referring to FIG. 1, the controller 104 may be configured to receive information from the auxiliary inputs 140 which may include environmental sensors, user inputs, and/or information from a lookup table regarding chemical characteristics from a set of fragrance characteristics. In some embodiments, the environmental sensors may comprise the sensors listed below that may detect the cartridge or the type of volatile material within the cartridge. The controller 104 may include a processor that processes the information to determine an optimal power level for each of the discrete fragrance characteristic switching levels, i.e., an optimal heating temperature can be determined to produce a desired scent. In addition, the controller 104 may determine a power level to be sent to the air displacement mechanism 116 to produce a desired airflow.

The environmental sensors that may be used may include, but are not limited to, a microphone, a camera, a turbidity sensor, a thermometer, a humidity sensor, a passive infrared sensor, a light sensor, a lightning sensor, a wind transducer, a compass, a Global Positioning System (“GPS”), a gyroscope, an accelerometer, a barometer, a crash sensor, a proximity sensor, a radar, an ultrasonic sensor, or any combination thereof. The chemical characteristics of the volatile material may be measured by the volatile material dispenser 100 or transmitted to the controller 104 through a wireless communication device or other means. Some of the user inputs may include but are not limited to time preferences for operation, intensity level, and fragrance selection. In some embodiments, the controller 104 receives the information from the environmental sensor(s), the user inputs, and the chemical characteristics of the volatile material, and regulates the volatile material dispenser 100 to achieve a desired heating temperature of the heating element and a desired airflow of the air displacement mechanism 116. Thus, the desired airflow and temperature correlate with a desired volatile material concentration level in the surrounding environment.

In some embodiments, the auxiliary inputs 140 may include user inputs which may be input into the volatile material dispenser 100 via external switches. In some embodiments, a first switch may be configured to control an intensity of the fragrance. A first switch setting may define a “low” setting, which may set the heating element 120 to operate with 5 minutes between each operational cycle. A second switch setting may define a “medium” setting that may set the heating element 120 to operate with 3 minutes between each operational cycle. A third switch setting may define a “high” setting that may set the heating element 120 to operate with 1 minute between each operational cycle. By adjusting the first switch between the three or more switch settings, the timing between operational cycles differs so that different fragrance intensities and volatile material intensities can be achieved in the surrounding environment. In some embodiments, there may be more than one switch. For example, there may be a second switch, that allows a user to control a fragrance output of volatile material, e.g., apple, apple/cinnamon, cinnamon. In some embodiments, the first switch and the second switch may additionally or alternatively be provided via a display screen of an electronic device that is in communication with one or more of the electrical components within the volatile material dispenser 100.

In some embodiments, the volatile material dispenser 100 may include features that provide enhanced customization and adaptation capabilities. Algorithms may be used by the volatile material dispenser 100 to modify the operational parameters according to user preferences and/or volatile material requirements. Generally, the controller 104 operates the volatile material dispenser 100 according to pre-programmed sequences, which are designed to control the temperature of the heating element within 1° F. (0.55° C.) of the targeted temperature. More specifically, algorithms may be used to vary the power applied to the heater, as well as the output of the air displacement mechanism 116 (e.g., controlling the speed or revolutions per minute (RPM) of the air displacement mechanism 116). For example, PWM may be used to adjust the heating element 120 to different temperatures according to various duty cycles.

Controlling the temperature of the heating element 120 and rate of air displacement may result in controlling evaporated volatile material 128 intensities and fragrance characteristics of the evaporated volatile materials 128, which creates a consistent volatile material intensity and fragrance experience despite the particular user environment. The operation of the volatile material dispenser 100 may be adjusted directly through user manipulated controls or wirelessly through an electronic device, such as a user's mobile device. In some embodiments, the heating element 120 is configured to achieve a temperature of between about 100° F. and about 300° F., or between about 125° F. and about 275° F., or between about 150° F. and about 250° F., or between about 175° F. and about 225° F. The heating element 120 may comprise one or more resistors having a resistance of between about 0.5 Ohms (Ω) and about 50.0 Ω, or between about 1.0 Ω and about 15.0 Ω, or between about 1.5 Ω and about 10.0 Ω, or between about 2.5 Ω and about 7.5 Ω, or between about 3.5 Ω and about 5.0 Ω.

In some embodiments, the power limits may vary for different volatile materials or different environments. Further, the intensity limits (e.g., on time/off time between activations) may also be different among different volatile materials and environments. In some embodiments, both duration and intensity limits may be changed throughout operation of the volatile material dispenser 100 due to changes that may occur over time and/or due to one or more of a number of external factors that may be measured or identified either automatically or from information input by a user. In some embodiments, automatic triggers may cause the duration limits or intensity limits (e.g., minimum and maximum switch positions) to be adjusted, for example, based on a time of the day, a measured room temperature, a season of the year, or vital signs of a person acquired from the electronic device (e.g., a cell phone, a smart watch, or another type of device having sensors or that can receive user inputs). The user inputs may be received from environmental sensors, which may include external sensors along any number of devices that are configured to communicate either directly with the volatile material dispenser 100, or with the electronic device, and that are configured to provide feedback to the volatile material dispenser 100. The changes in intensity levels can also be triggered via request from an app or via direct physical interaction with the volatile material dispenser 100.

In some embodiments, the fragrance characteristics and volatile material characteristics can be enhanced through different durations of operation and/or different frequencies of operation. For example, different frequencies of operation could include the volatile material dispenser 100 operating a duty cycle every 5 minutes or every 15 minutes. In some embodiments, the fragrance characteristics and volatile material characteristics can be enhanced through an algorithm that varies the temperature of activation in a predetermined fashion and/or through the use of an on/off timer. In some embodiments, the varying of a speed of the air displacement mechanism 116 when the heating element 120 is turned “on” is used to enhance the characteristics of the volatile material 128. In some embodiments, a user is capable of controlling the various parameters discussed above through an app, which allows a user to vary characteristics of the volatile material by choosing different pre-determined operational points that are connected to volatile material characteristics such as a fragrance. In some embodiments, the operational points may be pre-determined or pre-programmed.

In some embodiments, the refill 124 includes a cartridge having embedded information (e.g., along an outer side thereof) which may be automatically detected via one or more sensors within the volatile material sensor. To that end, the volatile material dispenser 100 may include one or more sensors 144, such as an infrared (IR) sensor, an optical sensor, a weight sensor, a hall effect sensor, one or more magnets, a radio frequency identification (RFID) sensor, a barcode scanner, a QR scanner, a proximity sensor, a reflective photo interpreter, a humidity sensor, a fluid property sensor, a light sensor, an alcohol sensor, or another type of sensor. In some embodiments, the volatile material dispenser 100 may be configured to retrieve data that provides information regarding the collected sensory input from the sensors 144. For example, the volatile material dispenser 100 may be configured to read a QR code (e.g., via a camera) that may scan a QR code along an exterior of the refill 124. The QR code may be associated with embedded information including anticipated humidity level, pressure levels, temperature, or any other type of information that may influence the timing of the run cycle(s) and the heating intensity of the heating element 120. In some embodiments, the volatile material may be produced for or configured for volatilization in a tropical environment. The volatile material dispenser 100 may then vary the temperature of the heating element 120 based on receiving information from the refill 124 that the volatile material is likely in a particular environment such as a tropical environment.

In some embodiments, data from one or more of the above-referenced sensors 144 may be received by the controller 104. The controller 104 may be configured to have a memory, a processor, and information stored within a lookup table that may be compared against data that is retrieved by any of the sensors. An identified fragrance or fragrance characteristic of the volatile material 128 may be determined based on the comparison of the data with the information stored within the lookup table. However, in some embodiments a user may manually input information that identifies a refill type or the environmental conditions. In some embodiments, the refill 124 is a cartridge that includes two fragrances that are configured to release two different fragrance characteristics, i.e., the cartridge is a 2-tone cartridge. In some embodiments, the volatile material may be identified based on data that is retrieved by the one or more sensors noted above in combination with data manually input by a user.

In some embodiments, a lock-and-key feature is provided with the refill 124 in the form of a cartridge. For example, the lock-and-key feature may be a protrusion along an upper rim of the cartridge, which may provide information to the volatile material dispenser 100 regarding the volatile material within the cartridge. The lock-and-key feature may be disposed along the exterior surface of the cartridge or may be provided along an inner surface of the cartridge (e.g., along a channel defining a mouth of the cartridge). The volatile material dispenser 100 may be configured to detect a type of volatile material 128 that is within the refill 124 based on a location of the lock-and-key feature. For example, a cartridge filled with a volatile material that works well in the tropics may be identified based on a location of two radially offset protrusions along an exterior surface of the cartridge.

In some embodiments, the sensor 144 measurements and temperature measurements are associated with different, pre-determined wattages, which can be pre-determined based on an identified vapor pressure of each of the particular volatile materials to be emanated from the volatile material dispenser 100 that corresponds with a temperature that is achieved based upon a specified power setting, e.g., 5.0 W, 5.5 W, and 6.0 W. Although ranges of 5.0 to 6.0 W are discussed, various other heating profiles are possible (e.g., between 1.0 W and 9.0 W) and can be chosen based upon the volatile material present within the refill 124.

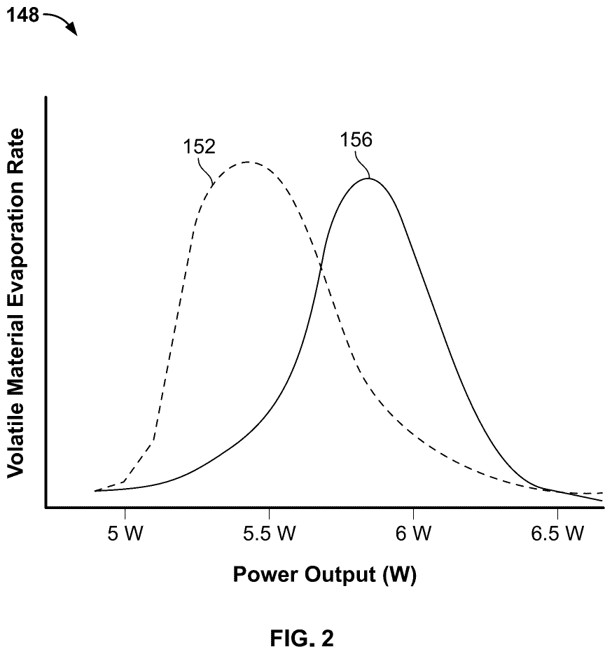

Referring to FIG. 2, a bi-modal graph 148 is provided showing differing evaporation rates for the same volatile material with the same volatile material dispenser 100 of FIG. 1 in two different environments. The first distribution 152 is of the volatile material evaporation rate of the volatile material 128 and the volatile material dispenser 100 being used in a hot, dry, desert like environment. The hot and dry environment may lead to a higher temperature of the wick 136, which lowers the amount of energy needed to evaporate the volatile material 128. Thus, the first distribution 152 is skewed closer 5.5 W of power output. The second distribution 156 is of the volatile material dispenser 100 being used in a cool, humid, artic environment. The cold and humid environment may lead to a lower temperature of the wick 136, which increases the amount of energy needed to evaporate the volatile material. Thus, the second distribution is skewed closer to 6 W of power output. The difference in evaporation rates can be attributed to both the performance of the volatile material dispenser 100 and the environmental factors that alter the evaporation rate of the volatile material 128 and the temperature of the wick 136.

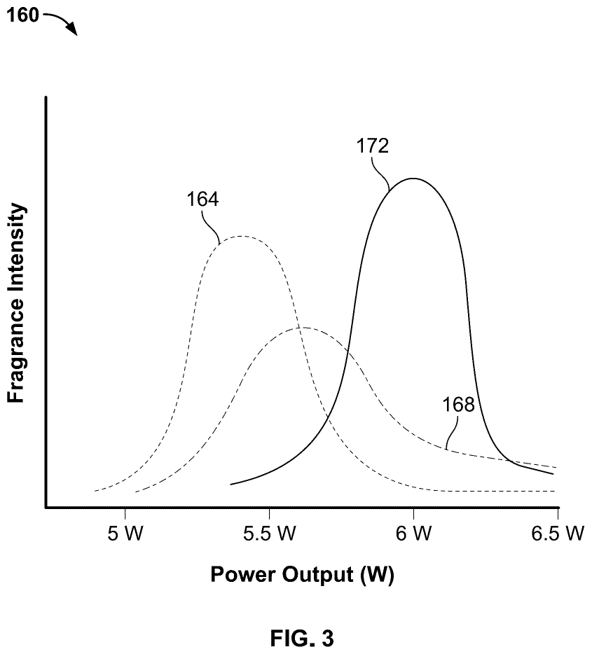

Referring to FIG. 3, a tri-modal accord 160 is illustrated for some example embodiments of the volatile material 128 of FIG. 1. In these embodiments, the volatile material 128 has three different fragrance characteristics that are emitted at different temperature and power outputs of the volatile material dispenser 100. The three different fragrances may be associated with “lavender,” “jasmine,” and “vanilla” fragrance characteristics, for example. In some embodiments, one or more additional fragrance characteristics may be included, and each fragrance characteristic may have a different optimal fragrance release temperature and power output of the volatile material dispenser 100, as shown by the peaks within the graph of FIG. 3. In FIG. 3, the first fragrance characteristic 164 smells more like “lavender” with a hint of “jasmine” and “vanilla,” the second fragrance characteristic 168 smells more like “jasmine” with more subtle notes of “lavender” and “vanilla,” and the third fragrance characteristic 172 smells more like “vanilla” with hints of “jasmine” and “lavender.” Still referring to FIG. 3, the optimal power output of the volatile material dispenser 100 for the first fragrance characteristic 164 is about 5.4 W because the power output of about 5.4 W corresponds with the highest fragrance intensity of “lavender,” the first fragrance characteristic 164. Further, the optimal power output of the volatile material dispenser 100 for the second fragrance characteristic 168 is about 5.6 W because the power output of about 5.6 W corresponds with the highest fragrance intensity of “jasmine,” the second fragrance characteristic 168. Additionally, the optimal power output of the volatile material dispenser 100 for the third fragrance characteristic 172 is about 6.0 W because the power output of about 6.0 W corresponds with the highest fragrance intensity of “jasmine,” the third fragrance characteristic 172. More fragrance characteristics may be included, and additional, non-fragranced compositions may be included and configured for release within one or more temperature schemes, as described in greater detail below. In some embodiments, the optimal power output may correspond with the highest rates of volatile material evaporation.

Still referring to FIG. 3, the three different temperature and power outputs that correlate with the three different fragrance characteristics can fluctuate based on the temperature of the wick 136 and other environmental factors. The fluctuation of the three different temperature and power outputs that correlate with the three different fragrance characteristics can be similar to the fluctuations of evaporation rates that were shown in the bi-modal graph 148 of FIG. 2.

In some embodiments, the user may be able to control which fragrance the volatile material dispenser outputs by switching the volatile material dispenser 100 to a lower or first setting, that emits the first fragrance characteristic 164, by switching the volatile material dispenser to a middle or second setting, the user can emit the second fragrance characteristic 168, and by switching the volatile material dispenser to a higher or third setting, the user can emit a third fragrance characteristic 172. By allowing for three different fragrance characteristics to be emitted from the volatile material dispenser based on a selected input from a user, the volatile material dispenser is configured to provide a user the ability to select, within pre-determined minimum and maximum limits, how a fragrance will smell.

It should be noted that intensity selection is (e.g., an intensity selection of high, medium, or low) different than an amount of time of activation and/or an amount of time between activations or operation cycles. The fragrance characteristic selection allows an end user to be able to select a fragrance characteristic. In the illustrated embodiments of FIG. 3, the first, second, and third fragrance characteristics 164, 168, 172 are associated with different, pre-determined wattages, which can be pre-determined based on an identified vapor pressure of each of the particular fragrances to be emanated from the volatile material dispenser that corresponds with a temperature that is achieved based upon a specified power output setting, e.g., 5.0 W, 5.5 W, and 6.0 W. In some embodiments, the second switch may include discrete positions such that only a pre-determined number of fragrance characteristics may be implemented, e.g., 2, 3, 4, etc. However, in alternative embodiments, an infinite number of fragrance characteristics may be implemented. To that end, the second switch may be a dial that is movable between a minimum setting and a maximum setting and may be adjustable to achieve an infinite number of temperature settings. Although ranges of 5.0 to 6.0 W are discussed, various other heating profiles are possible (e.g., between 1.0 W and 9.0 W) and can be chosen based upon the fragrance present within the refill 124.

In some embodiments, the first fragrance characteristic 164 is achieved using a temperature scheme among one of the following temperature schemes, although varying power schemes may also be utilized depending on the type of fragrance to be emitted. In some embodiments, the first fragrance characteristic 164 is achieved at a temperature within a first range of between about 150° F. and about 200° F., or between about 160° F. and about 190° F., or about 175° F. In some embodiments, the second characteristic 168 is achieved at a temperature within a second range of between about 175° F. and about 225° F., or between about 185° F. and about 215° F., or about 200° F. In some embodiments, the third fragrance characteristic 172 is achieved at a temperature within a third range of between about 200° F. and about 250° F., or between about 210° F. and about 240° F., or about 225° F. In some embodiments, the optimal temperature schemes are influenced by the temperature of the wick 136. For example, if the wick 136 has a temperature of 65° F. the temperature schemes may be increased to achieve the desired fragrance characteristics. In contrast, if the temperature of the wick 136 is 95° F. the temperature schemes may be decreased to achieve the desired fragrance characteristics. In some embodiments, the temperature ranges between the first fragrance characteristic 164, the second fragrance characteristic 168, and the third fragrance characteristic 172 may overlap. In such embodiments, overlapping portions of the ranges may denote two of the fragrances being identifiable by a user. In other embodiments, the temperature ranges between the first fragrance characteristic 164, the second fragrance characteristic 168, and the third characteristic 172 do not overlap.

For example, the first fragrance characteristic 164 may be achieved at a temperature within the first range of between about 150° F. and about 200° F., the second fragrance characteristic 168 may be achieved at a temperature within the second range of between about 175° F. and about 225° F., and the third fragrance characteristic 172 may be achieved at a temperature within the third range of between about 200° F. and about 250° F. In some embodiments, a fragrance profile may be identified to align with a pre-defined temperature scheme of the volatile material dispenser 100, e.g., 175° F., 200° F., and 225° F. To that end, a first fragrance may be identified that has a fragrance characteristic that is achieved within the first temperature range, a second fragrance may be identified that has a fragrance characteristic that is achieved within the second temperature range, and a third fragrance may be identified that has a fragrance characteristic that is achieved within the third temperature range. Additionally, the first fragrance may have a peak or desired first fragrance characteristic at a first temperature, the second fragrance characteristic may have a peak or desired second fragrance characteristic that is different than the first fragrance characteristic at a second temperature, and the third fragrance may have a peak or desired third fragrance characteristic that is different than the first and second fragrance characteristics at a third temperature.

In some embodiments, there may be a second switch allows a user to choose a particular power output that allows for the fragrance characteristics to be achieved that are associated with a first composition, a second composition, and a third composition. The first composition defines the first fragrance characteristic 164 (e.g., “lavender”), the second composition defines the second fragrance characteristic 168 (e.g., “jasmine”), and the third composition defines the third fragrance characteristic 172 (e.g., “vanilla”). The fragrance characteristics 164, 168, 172 may be associated with a peak in fragrance intensity of the chemical compositions or the fragrance characteristics may be associated with relative increases in fragrance intensities. In some embodiments, the chosen particular power output may be associated with a particular unique mix of the fragrance characteristics, such that the particular power output may correspond to the particular mix of smells that smell more like “jasmine” and “lavender” with subtle notes of “vanilla.” In some embodiments, the particular power output may correspond with a particular volatile material evaporation rate. In some embodiments, the particular power output is the optimal power output for the highest rate of volatile material evaporation and/or the highest fragrance intensity. In some embodiments, the particular power output may correspond with a particular measurement of the temperature of the wick 136.

In some embodiments, the controller 104 may choose what optimum temperature the heating element 120 should be for each cycle. In some embodiments, the controller 104 may vary the power output to the heating element 120 in each cycle to vary the fragrance characteristics of the evaporated volatile material 128 in each cycle. In some embodiments, the scents may be in a random order to prevent a user from becoming accustomed to or habituated to a particular volatile material because the perception of a scent by a user of a dispensed fragrance at a constant intensity tends to decay over time. In some embodiments, the controller 104 may have a predetermined order of power outputs that can correlate with the season or time of day. In some embodiments, the controller 104 uses a randomizer to determine what power output and fragrance characteristic should be discharged. In some embodiments, the volatile material dispenser 100 may not have varying cycles that correlate with different fragrance characteristics.

In some embodiments, the fragrance characteristics are associated with peak fragrance intensities of the respective compositions, or only some of the fragrance characteristics are associated with peak fragrance intensities. Still further, in some embodiments, the fragrance characteristic may be associated with a non-fragranced active, such as a pest control active, which may be volatized at a higher rate than other compositions within the refill 124. In such an embodiment, the fragrance characteristics are reflective of an attribute or characteristic of the composition that is non-fragrance based.

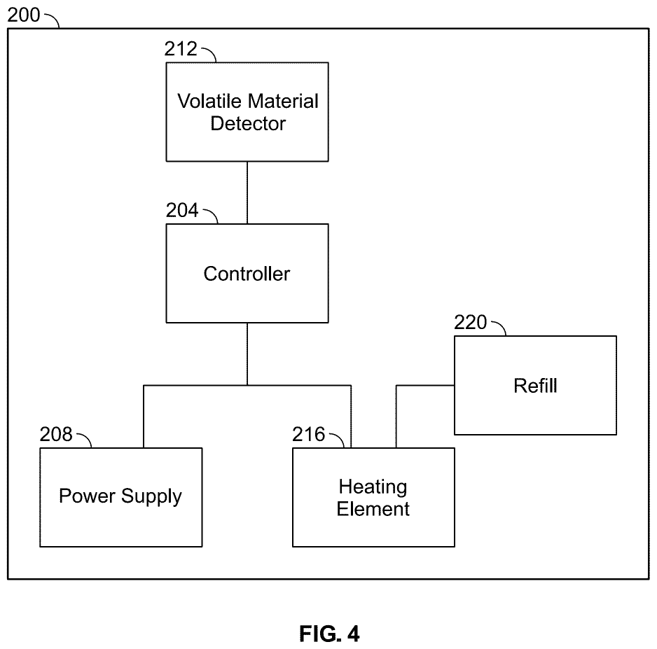

Now referring to FIG. 4, a volatile material dispenser 200 is shown. The volatile material dispenser 200 comprises a controller 204 that is in communication with a power supply 208, a volatile material detector 212, and a heating element 216. The heating element 216 is in communication with a refill 220. It will be appreciated that the volatile material dispenser 200 of FIG. 2 includes components that are similar, i.e., structurally and/or functionally similar, with components of the volatile material dispenser 100 of FIG. 1. For example, the controller 204 of FIG. 2 may be similar to the controller 104 of FIG. 1. Additionally, the heating element 216 may be similar to the heating element 120 of FIG. 1. The power supply 208 and the refill 220 may be similar to the power supply 108 and the refill 124 of FIG. 1.

The volatile material dispenser 200 of FIG. 4 may be configured to use the volatile material detector 212 to monitor the volatile material concentration and/or the fragrance characteristics of the evaporated volatile material in the surrounding environment. The volatile material dispenser 200 may be configured to run an initial run cycle upon being turned on by a user. The initial run cycle may be configured to run the volatile material dispenser 200 until the volatile material detector 212 measures a predetermined threshold of a volatile material concentration and/or a concentration of a specific volatile material fragrance characteristic in the surrounding environment. After the predetermined volatile material concentration and/or a concentration of a specific volatile material fragrance characteristic is measured, the volatile material dispenser 200 may then fall into a secondary cycle (i.e., a sustaining cycle, after an initial filling cycle). The secondary cycle may include the volatile material dispenser 200 running on high for 10 seconds every two minutes, running on high for 5 seconds every 5 minutes, or running on high for 10 seconds every 10 minutes.

In some embodiments, the volatile material dispenser 200 may additionally or alternatively have a secondary cycle that corresponds with a ratio of time running on high to rest time. For example, the volatile material dispenser 200 may have a “low” setting where the volatile material dispenser 200 runs for about 1 unit of time followed by a rest period of between about 150 and about 250 units of time, a “medium” setting where the volatile material dispenser 200 runs on high for about 1 unit of time followed by a rest period of between about 75 and about 150 units of time, and a “high” setting where the volatile material dispenser 200 runs for about 1 unit of time followed by a rest period of between about 25 and about 75 units of time. In some embodiments, the secondary cycle may include the volatile material dispenser 200 being a rest period until being activated by a motion sensor. In some embodiments, a unit of time may be a microsecond, a millisecond, a second, a minute, an hour, a day, or any other unit of time. Any of the running cycles disclosed herein may be initiated by a period of user detection using a motion sensor such as an infrared (“IR”) sensor.

Still referring to FIG. 4, the volatile material dispenser 200 may be configured to run an initial run cycle upon being turned on by a user. The initial run cycle may include the volatile material dispenser 200 running until the volatile material detector 212 measures a predetermined threshold of a volatile material concentration and/or a concentration of a specific volatile material fragrance characteristic in the surrounding environment. After the predetermined volatile material concentration and/or the concentration of a specific volatile material fragrance characteristic is measured, the volatile material dispenser 200 may be configured to only run after the volatile material detector 212 measures a volatile material concentration and/or a concentration of a specific volatile material fragrance characteristic that are below a predetermined or user-input threshold. In some embodiments, the volatile material dispenser 200 may run until the volatile material concentration and/or the concentration of a specific volatile material fragrance characteristic is measured at or above a predetermined threshold. Thus, the volatile material concentration and/or the concentration of a specific volatile material fragrance characteristic within the environment may remain within a range pertaining to a predetermined or user-input volatile material concentration and/or a concentration for a specific volatile material fragrance characteristic.

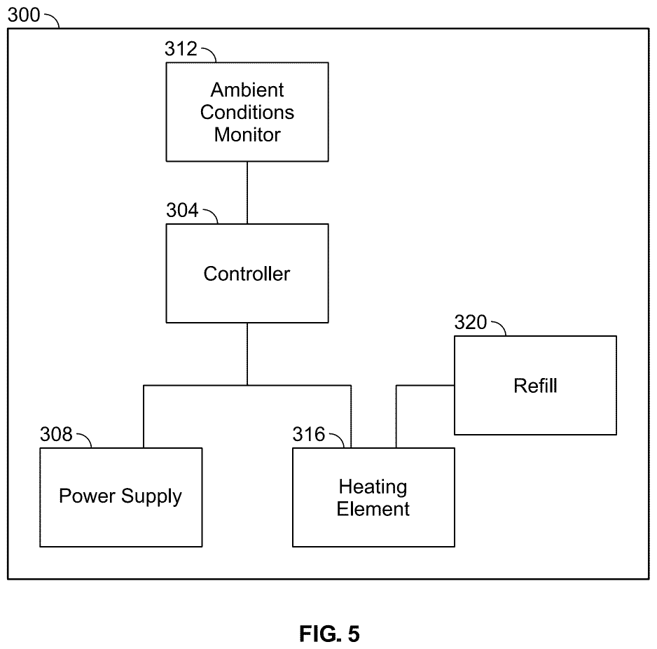

Now referring to FIG. 5, a volatile material dispenser 300 is shown. The volatile material dispenser 300 comprises a controller 304 that is in communication with a power supply 308, an ambient conditions monitor 312, and a heating element 316. The heating element 316 is in communication with a refill 320. The volatile material dispenser 300 of FIG. 5 includes components that are similar, i.e., structurally and/or functionally similar, with components of the volatile material dispenser 100 of FIG. 1. For example, the controller 304 of FIG. 5 may be similar to the controller 104 of FIG. 1. Additionally, the heating element 316 may be similar to the heating element 120 of FIG. 1. The power supply 308 and the refill 320 may be similar to the power supply 108 and the refill 124 of FIG. 1.

The volatile material dispenser 300 of FIG. 5 may be configured to monitor the air flow in an environment using the ambient conditions monitor 312 that is in communication with the controller 304. The volatile material dispenser 300 may be configured to determine if there is a high level of air flow using the ambient conditions monitor 312. If the ambient conditions monitor 312 communicates to the controller 304 that there is a high level of air flow, the controller 304 may be configured to shut off the volatile material dispenser 300. In some embodiments where the volatile material dispenser 300 is located in a house or an office building, a high level of air flow may be the result of air flow from a heating, ventilation, and air conditioning system (“HVAC system”). If the HVAC system is on, the air may be circulated outside of the house or the office and/or passed through a filter. Thus, if the volatile material dispenser 300 is turned on while there is a high level of air flow from an HVAC system, the dispenser 300 may have reduced efficiency in reaching a desired volatile material concentration in the surrounding environment since the evaporated volatile material may be taken away and/or filtered out at a faster rate. In some embodiments, the volatile material dispenser 300 may be configured to activate immediately after a period of high air flow detected by the ambient conditions monitor 312. By activating immediately after a cycle of measured high air flow, the volatile material dispenser 300 may be able to raise the volatile material concentration back to the predetermined volatile material concentration threshold in the surrounding environment.

In some embodiments, the volatile material dispenser 300 may be configured to determine if there is a high level of air flow using the ambient conditions monitor 312. If the ambient conditions monitor 312 communicates to the controller 304 that there is a high level of air flow, the controller 304 may be configured to turn on the volatile material dispenser 300 to take advantage of the air displacement caused by the HVAC system to diffuse the volatile material to desired locations within the surrounding environment. The ambient conditions monitor 312 may comprise one or more of the sensors discussed above, as well as any other device or combination of devices capable of identifying an environmental characteristic or parameter. In some embodiments, the ambient conditions monitor 312 may be configured to monitor one or more ambient conditions that may include an ambient temperature, an ambient humidity level, an ambient air flow level, an ambient air pressure, an ambient ultraviolent (“UV”) index, and/or any other ambient condition. Further, the ambient conditions monitor 312 may be configured to monitor the ambient conditions by receiving information from an outside source such as a smart home device regarding the ambient conditions. In other embodiments, the ambient conditions monitor 312 receives information from one or more sensors or devices local to the monitor 312 and the volatile material dispenser 300 as well as one or more ambient conditions from an outside source. In some embodiments, the ambient conditions monitor 312 may measure and/or receive data pertaining to one or more ambient conditions such as the ambient temperature. The ambient conditions monitor 312 may then send the data to the controller 304 such that the controller 304 can determine and output a corresponding optimal or desirable output power to the heating element 316 based on the ambient conditions.

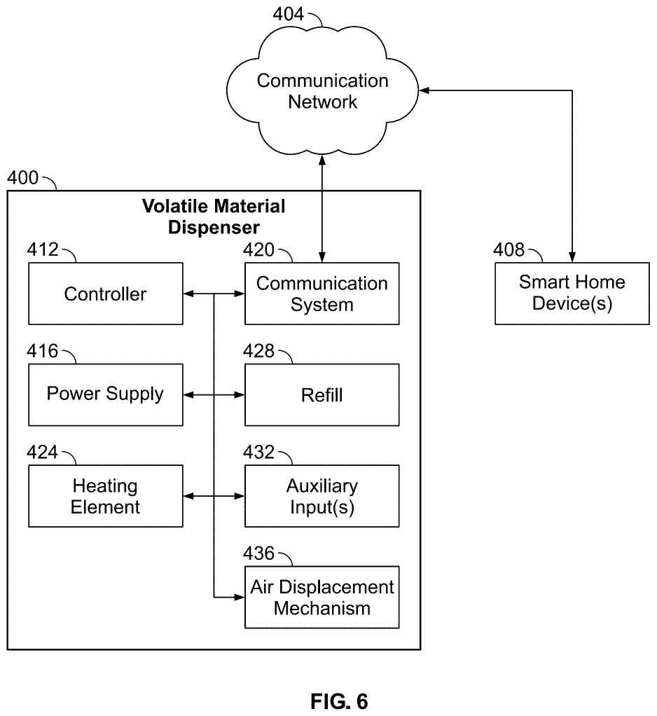

Now referring to FIG. 6, a volatile material dispenser 400 in communication with a communication network 404 is shown. The communication network 404 is in communication with one or more smart home devices 408 and the volatile material dispenser 400. The volatile material dispenser 400 comprises a controller 412, a power supply 416, a communication system 420, a heating element 424, a refill 428, one or more auxiliary inputs 432, and an air displacement mechanism 436. It will be appreciated that the volatile material dispenser 400 of FIG. 6 includes components that are similar (i.e., structurally and/or functionally similar) with respect to components of the volatile material dispenser 100 of FIG. 1. For example, the controller 412 of FIG. 6 may be similar to the controller 104 of FIG. 1. Additionally, the heating element 424 may be similar to the heating element 120 of FIG. 1. The power supply 416 and the refill 428 may be similar to the power supply 108 and the refill 124 of FIG. 1.

Still referring to FIG. 6, the one or more smart home devices 408 that are in communication with the communication network 404 may include but are not limited to a Google® Nest® Thermostat, a home security system, an Amazon® Alexa®, a Ring® Video Doorbell, a motion detector, a garage door opener, a gate access opener, a neighborhood guardhouse, a smart T.V., a thermostat, an automatic lock, an HVAC unit, and/or any other smart home device. In some embodiments, the smart home devices 408 may include a wireless communication device such as a mobile phone with a graphical user interface (GUI), or any other device, such as, e.g., a laptop, a tablet, a desktop, a server, or a special purpose computer.

The communication network 404 can be any suitable communication network or combination of communication networks. For example, the communication network 404 may include a Wi-Fi network (which can include one or more wireless routers, one or more switches, etc.), a peer-to-peer network (e.g., a Bluetooth® network), a cellular network (e.g., a 3G network, a 4G network, a 5G network, etc., complying with any suitable standard, such as CDMA, GSM, LTE, LTE Advanced, NR, etc.), a wired network, etc. In some embodiments, the communication network 404 can be a local area network (LAN), a wide area network (WAN), a public network (e.g., the Internet), a private or semi-private network (e.g., a corporate or university intranet), any other suitable type of network, or any suitable combination of networks.

Communications links shown in FIG. 6 can each be any suitable communications link or combination of communications links, such as wired links, fiber optic links, Wi-Fi links, Bluetooth® links, cellular links, etc. In a preferred embodiment, the communication network 404 is a Bluetooth® network that is compatible with all the components of the volatile material dispenser 400. The communications network systems (not shown) that are used to comprise the communication network 404 can include any suitable hardware, firmware, and/or software for communicating information over the communication network 404 and/or any other suitable communication networks 404. For example, the communication network systems (not shown) can include one or more transceivers, one or more communication chips and/or chip sets, etc. In a more particular example, the communication network systems (not shown) include hardware, firmware and/or software that can be used to establish a Wi-Fi connection, a Bluetooth® connection, a cellular connection, an Ethernet connection, etc.

The communication system 420 in the volatile material dispenser 400 can be any communication device such as a special purpose computer. The communication system 420 may support Bluetooth® Low Energy (BLE) wireless communication, Wi-Fi, or other types of wireless communication. In a preferred embodiment, the communication system 420 may include one or more of onboard crystal oscillators, chip antenna, and/or passive components. The communication system 420 may support a number of peripheral functions such as ADC, timers, counters, PWM, and serial communication protocols, (e.g., I2C, UART, SPI), through its programmable architecture. The communication system 420 may be an electronic device that includes a processor, a flash memory, a timer, and additional components typical of such electronic devices. The communication system 420 may be used to collect and transmit information through the wireless communication network 404 to be delivered to the controller 412. For example, the information can include user preferences, switch selection, and environmental factors and measurements. In some embodiments, the communication system 420 collects data associated with environmental factors and measurements using environmental sensors via the auxiliary inputs 432. The environmental sensors may include any of the sensors discussed above.

Referring to FIG. 6, the communication system 420 may send the user inputs and auxiliary inputs 432 to the wireless communication network 404 to be relayed to the smart home devices 408. In some embodiments, the smart home devices 408 may send the user inputs and the auxiliary inputs 432 to the communication network 404 to be sent to the communication system 420 and the controller 412 of the volatile material dispenser 400. In some embodiments, the user may directly input data corresponding to the auxiliary inputs 432 via a graphical user interface (GUI) provided on a display (not shown) and aided by memory (not shown) as part of one of the smart home devices 408. In some embodiments, the display (not shown) includes any suitable display devices, such as a computer monitor, a touchscreen, a television, a projector, etc. In some embodiments, any suitable input devices and/or sensors can be used to generate the user inputs for the auxiliary inputs 432, such as a keyboard, a mouse, a touchscreen, a microphone, etc.

In some embodiments, the user may scan a QR code on the side of the refill 428 using the smart home device 408 such as a mobile phone. The smart home device 408 may then send information pertaining to the optimal power output cycles to the heating element 424 that is from the QR code to the volatile material dispenser 400 using BLE or a Wi-Fi network. In some embodiments, the user may input information that describes the current geographic location of the device and/or the current atmospheric conditions into the smart home device 408 (e.g., a mobile phone). The inputted information from the user could then be sent to the controller 412 to be used to determine the optimal power output cycles or the smart home device 408 may use the information to determine the optimal power output cycles and send the optimal power output cycles to the controller 412.

Still referring to FIG. 6, the volatile material dispenser 400 may receive information on the HVAC operating cycle through one or more of the smart home devices 408. In some embodiments, the volatile material dispenser 400 can shut itself off while the HVAC system is operating. After the HVAC system is done with its cycle of being on, the volatile material dispenser 400 may be configured to run an initial cycle to achieve a predetermined volatile material concentration threshold within the surrounding environment before proceeding to a secondary cycle where the volatile material concentration threshold is maintained. In some embodiments, the volatile material dispenser 400 can be configured to be placed inside of the air vent of a HVAC system in both residential and commercial settings. In some embodiments where the volatile material dispenser 400 is placed in an air vent of a HVAC system, the volatile material dispenser 400 may be configured to operate when the HVAC is operating and use it as the air displacement mechanism 436.

Still referring to FIG. 6, the volatile material dispenser 400 may receive information on the HVAC operating cycle through one or more smart home devices 408. In some embodiments, the volatile material dispenser 400 may be configured to turn on while the HVAC system is operating to make use of the airflow provided by the HVAC system as the air displacement mechanism 436. In these embodiments, the volatile material dispenser may or may not be located in an air vent.

Still referring to FIG. 6, the volatile material dispenser 400 may be configured to receive information related to the ambient temperature received from the smart home device 408, which may be a thermostat, and the controller 412 may be configured to adjust the power output to the heating element 424 based on the ambient temperature received from the smart home device 408. Further, the controller 412 may be configured to only apply the ambient temperature from a smart home device 408 and may not take into account the temperature of the refill 428 and/or the temperature of the heating element 424 when determining and outputting the output power to the heating element 424.

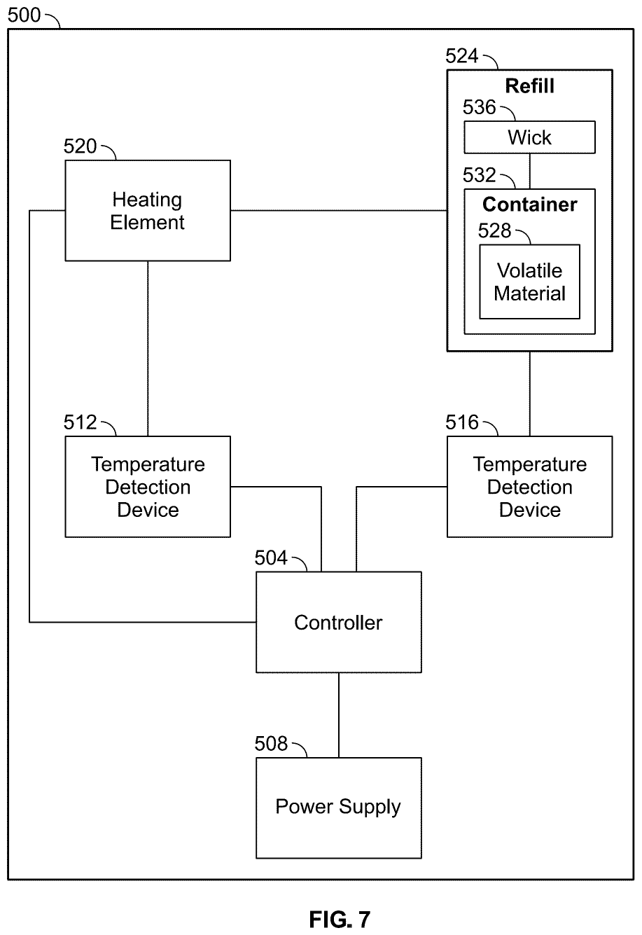

Now referring to FIG. 7, a volatile material dispenser 500 is shown. The volatile material dispenser 500 comprises a controller 504 that is in communication with a power supply 508, two temperature detection devices 512, 516, and a heating element 520. The heating element 520 is in thermal communication with a refill 524. The refill 524 includes a volatile material 528 disposed within a container 532. The refill 524 also comprises a fluid delivery system, shown as a wick 536, that is in fluid communication with the volatile material 528 and thermal communication with the heating element 520. The first temperature detection device 512 and the second temperature detection device 516 are in communication with the controller 504. The first temperature detection device 512 is in thermal communication with the heating element 520. The second temperature detection device 516 is in thermal communication with the refill 524. In some embodiments, the second temperature detection device 516 is in thermal communication with the volatile material 528, the container 532, the wick 536, or a combination thereof. It will be appreciated that the volatile material dispenser 500 of FIG. 7 includes components that are similar, i.e., structurally and/or functionally similar, with components of the volatile material dispenser 100 of FIG. 1. For example, the controller 504 of FIG. 7 may be similar to the controller 104 of FIG. 1. Additionally, the heating element 520 may be similar to the heating element 120 of FIG. 1. The power supply 508 and the refill 524 may be similar to the power supply 108 and the refill 124 of FIG. 1.

The volatile material dispenser 500 of FIG. 7 may be configured to monitor the temperature of both the refill 524 and the heating element 520. The controller 504 can receive the measurements from both of the temperature detection devices 512, 516 to control the power output from the controller 504 to the heating element 520. In some embodiments, the controller 504 can lower the power output to the heating element 520 if the temperature of the refill 524 is above a certain threshold. In some embodiments, the controller 504 can increase the power output to the heating element 520 or increase the length of the on time for each cycle if the detected temperature of the refill 524 is low. In some embodiments, the controller 504 can reduce the power output to the heating element 520 if the detected temperature is above a predetermined threshold. Alternatively, the controller 504 can lower the power output to the heating element 520 if the measured temperature of the heating element 520 is above a certain threshold. By controlling the output power to the heating element 520 based on the temperature of the heating element 520 and the refill 524, the volatile material dispenser 500 may be able to more accurately control the evaporated volatile material output and the fragrance characteristics of the evaporated volatile material. In some embodiments, the temperature detection device 516 is in communication with the volatile material within the refill 524. In some embodiments, the temperature detection device 516 is in communication with the wick 536 that is part of the refill 524.

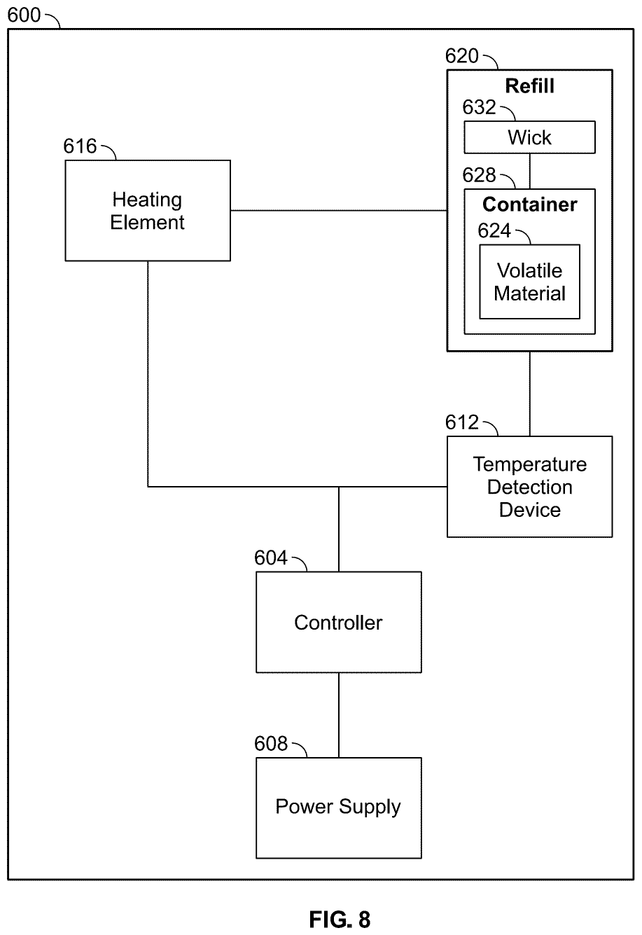

Now referring to FIG. 8, a volatile material dispenser 600 is shown. The volatile material dispenser 600 comprises a controller 604 that is in communication with a power supply 608, a temperature detection device 612, and a heating element 616. The heating element 616 is in thermal communication with a refill 620. The refill 620 includes a volatile material 624 disposed within a container 628. The refill 620 also comprises a wick 632 that is a fluid delivery system, and is in fluid communication with the volatile material 624 and thermal communication with the heating element 616. The temperature detection device 612 is in communication with the controller 604 and is in thermal communication with the refill 620. In some embodiments, the temperature detection device 612 is in thermal communication with one or a combination of the volatile material 624, the container 628, and/or the wick 632. It will be appreciated that the volatile material dispenser 600 of FIG. 8 includes components that are similar (structurally and/or functionally) with components of the volatile material dispenser 100 of FIG. 1. For example, the controller 604 of FIG. 8 may be similar to the controller 104 of FIG. 1. Additionally, the heating element 616 may be similar to the heating element 120 of FIG. 1. The power supply 608 and the refill 620 may be similar to the power supply 108 and the refill 124 of FIG. 1.

The volatile material dispenser 600 of FIG. 8 may be configured to monitor the temperature of the refill 620 as a whole, and/or the volatile material dispenser 600 of FIG. 8 may be specifically configured to monitor the temperature of one or more of the subcomponents of the refill 620, such as the wick 632. The controller 604 can receive the measurements from the temperature detection device 612 to control the power output from the controller 604 to the heating element 616. In some embodiments, the controller 604 can lower the power output to the heating element 616 if the temperature of the refill 620 or any subcomponent or combination of subcomponents of the refill 620 are above a certain threshold. In some embodiments, the controller 604 can increase the power output to the heating element 616 or increase the length of the “on” time for each cycle if the detected temperature of the refill 620 or any subcomponent or combination of the subcomponents of the refill 620 are below a predetermined threshold. By controlling the output power to the heating element 616 based on the temperature of the refill 620 or any of its subcomponents or a combination of the refill subcomponents, the volatile material dispenser 600 may be able to more accurately control the evaporated volatile material output and the fragrance experience of the evaporated scents. In some embodiments, the temperature detection device 612 is in communication with the volatile material 624 within the refill 620. In some embodiments, the temperature detection device is in thermal communication with only the wick 632.

In some embodiments, devices or systems disclosed herein can be used, manufactured, or installed using methods embodying aspects of the disclosure. Correspondingly, any description herein of particular features, capabilities, or intended uses of a device or system is generally intended to include disclosure of a method of using such devices for the intended purposes, of a method of otherwise implementing such capabilities, of a method of manufacturing relevant components of such a device or system (or the device or system as a whole), and of a method of installing disclosed (or otherwise known) components to support such purposes or capabilities. Similarly, unless otherwise indicated, discussion herein of any method of manufacturing or use for a particular device or system, including installing the device or system, is intended to inherently include disclosure, as embodiments of the disclosure, of the utilized features and configurations, and implemented capabilities of such device or system.

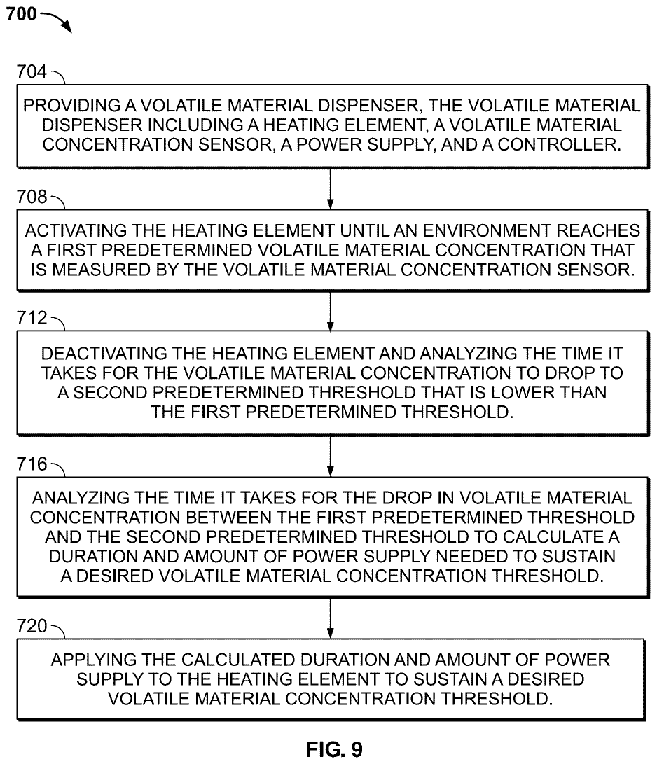

Referring now to FIG. 9, a method 700 is illustrated for emitting a volatile material, which may include fewer or more steps than depicted. At a first step 704, the method 700 includes providing a volatile material dispenser, the volatile material dispenser including a heating element, a volatile material concentration sensor, a power supply, and a controller. At a second step 708, the method 700 includes activating the heating element until the environment reaches a first predetermined volatile material concentration that is measured by the volatile material concentration sensor. At a third step 712, the method 700 includes deactivating the heating element and analyzing the time it takes for the volatile material concentration to drop to a second predetermined threshold that is lower than the first predetermined threshold. At a fourth step 716, the method 700 includes analyzing the time it takes for the drop in volatile material concentration between the first predetermined threshold and the second predetermined threshold to calculate a duration and amount of power supply needed to sustain a desired volatile material concentration threshold. At a fifth step 720, the method 700 includes applying the calculated duration and amount of power supply to the heating element to sustain a desired volatile material concentration threshold.

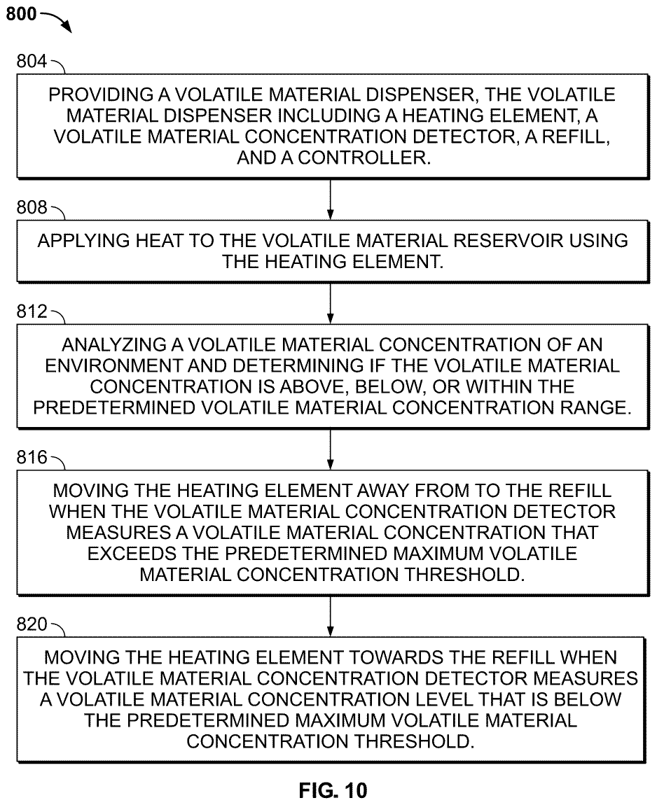

In FIG. 10, a method 800 for emitting the volatile material is shown, which may include fewer or more steps than depicted. At a first step 804, the method 800 includes providing a volatile material dispenser, the volatile material dispenser including a heating element, a volatile material concentration detector, a refill, and a controller. At a second step 808, the method 800 includes applying heat to the refill using the heating element. At a third step 812, the method 800 includes analyzing a volatile material concentration of an environment and determining if the volatile material concentration is above, below, or within the predetermined volatile material concertation range. At a fourth step 816, the method 800 includes moving the heating element away from the refill when the volatile material concentration detector measures a volatile material concentration that exceeds the predetermined maximum volatile material concentration threshold. At a fifth step 820, the method 800 includes moving the heating element toward the refill when the volatile material concentration detector measures a volatile material concentration that is below the predetermined maximum fragrance concentration threshold.

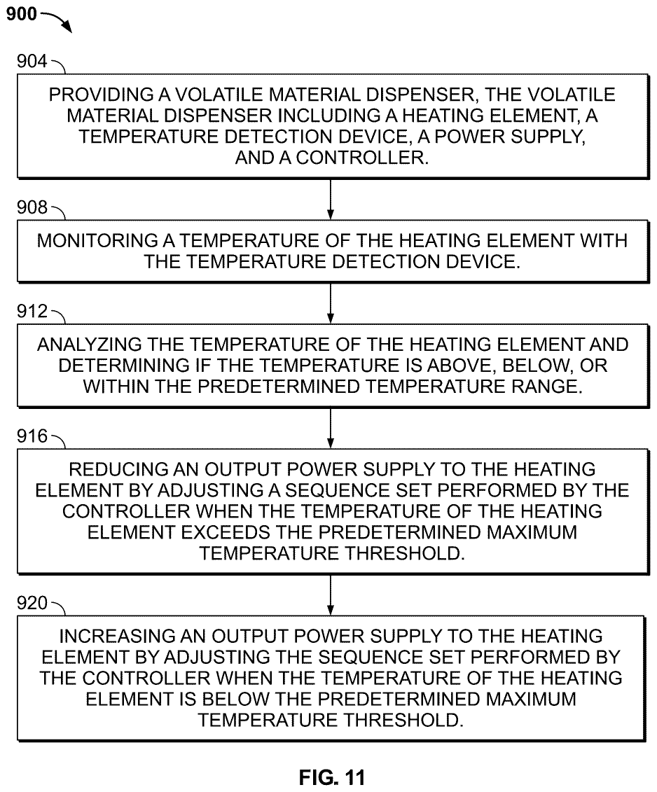

Referring now to FIG. 11, a method 900 is shown for emitting a volatile material, which may include more or fewer steps than depicted. At a first step 904, the method 900 includes providing a volatile material dispenser, the volatile material dispenser including a heating element, a temperature detection device, a power supply, and a controller. At a second step 908, the method 900 includes monitoring a temperature of the heating element with the temperature detection device. At a third step 912, the method 900 includes analyzing the temperature of the heating element and determining if the temperature is above, below, or within the predetermined temperature range. At a fourth step 916, the method 900 includes reducing an output power supply to the heating element by adjusting a sequence set performed by the controller when the temperature of the heating element exceeds the predetermined maximum temperature threshold. At a fifth step 920, the method 900 includes increasing an output power supply to the heating element by adjusting the sequence set performed by the controller when the temperature of the heating element is below the predetermined maximum temperature threshold.

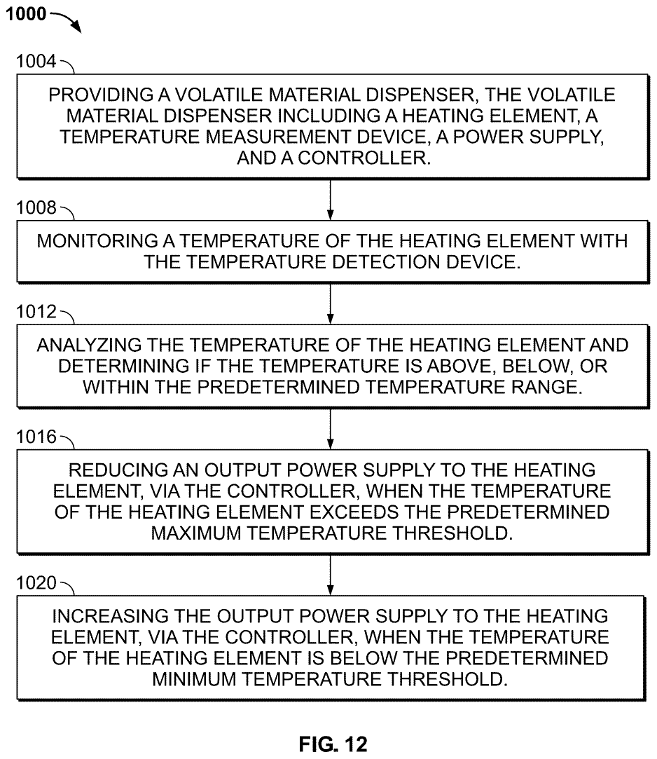

Referring now to FIG. 12, a method 1000 is shown for emitting a volatile material, which may include more or fewer steps than depicted. At a first step 1004, the method 1000 includes providing a volatile material dispenser, the volatile material dispenser including a heating element, a temperature measurement device, a power supply, and a controller. At a second step 1008, the method 1000 includes monitoring a temperature of the heating element with the temperature detection device. At a third step 1012, the method 1000 includes analyzing the temperature of the heating element and determining if the temperature is above, below, or within the predetermined temperature range. At a fourth step 1016, the method 1000 includes reducing an output power supply to the heating element, via the controller, when the temperature of the heating element exceeds the predetermined maximum temperature threshold. At a fifth step 1020, the method 1000 includes increasing the output power supply to the heating element, via the controller, when the temperature of the heating element is below the predetermined minimum temperature threshold.

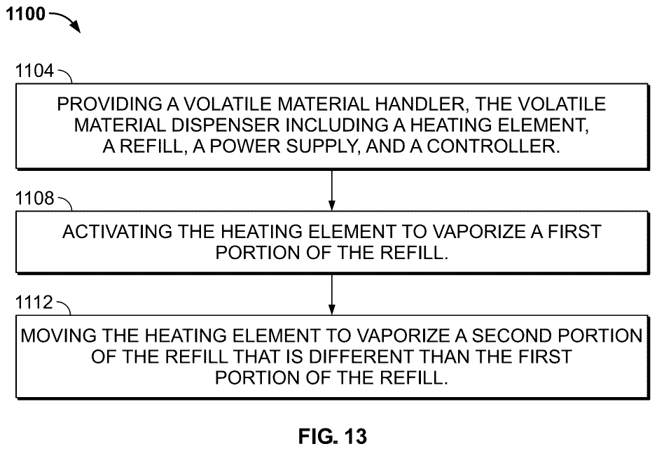

Referring now to FIG. 13, a method 1100 is shown for emitting a volatile material, which may include more or fewer steps than depicted. At a first step 1104, the method 1100 includes providing a volatile material dispenser, the volatile material dispenser including a heating element, a refill, a power supply, and a controller. At a second step 1108, the method 1100 includes activating the heating element to vaporize a first portion of the refill. At a third step 1112, the method 1100 includes moving the heating element to vaporize a second portion of the refill that is different than the first portion of the refill.

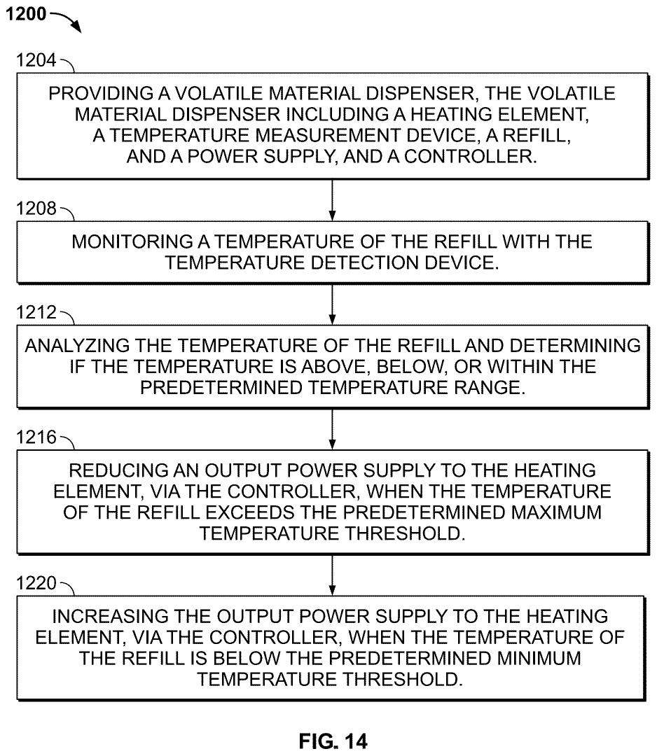

Referring now to FIG. 14, a method 1200 is shown for emitting a volatile material, which may include more or fewer steps than depicted. At a first step 1204, the method 1200 includes providing a volatile material dispenser, the volatile material dispenser including a heating element, a temperature measurement device, a refill, a power supply, and a controller. At a second step 1208, the method 1200 includes monitoring a temperature of the refill with the temperature detection device. At a third step 1212, the method 1200 includes analyzing the temperature of the refill and determining if the temperature is above, below, or within the predetermined temperature range. At a fourth step 1216, the method 1200 includes reducing an output power supply to the heating element, via the controller, when the temperature of the refill exceeds the predetermined maximum temperature threshold. At a fifth step 1220, the method 1200 includes increasing the output power supply to the heating element, via the controller, when the temperature of the refill is below the predetermined minimum temperature threshold. In some embodiments, the refill comprises a wick and a volatile material that is disposed within a container. In some embodiments, the temperature of the refill monitored by the temperature detection device is based on the measured temperature of the wick.

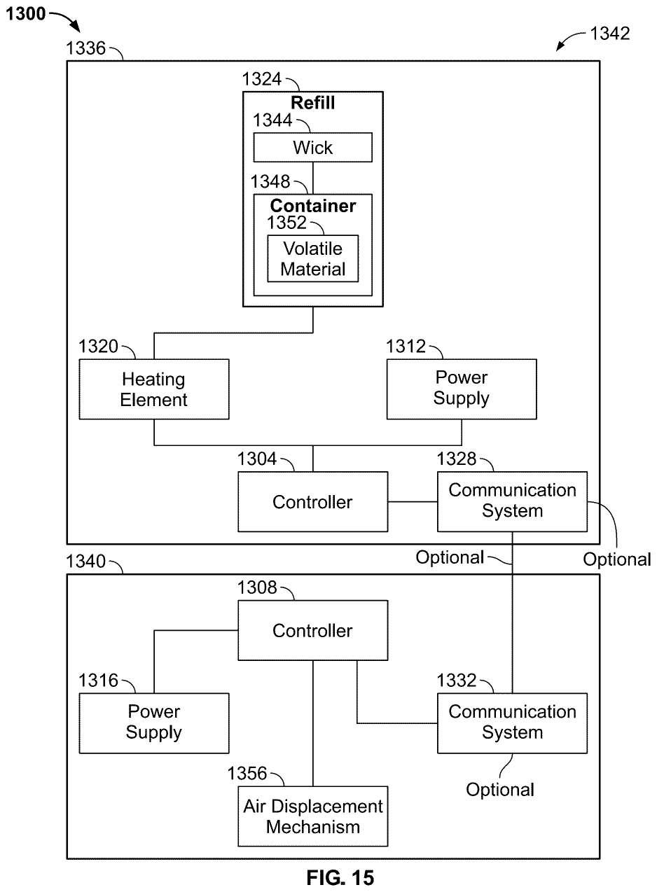

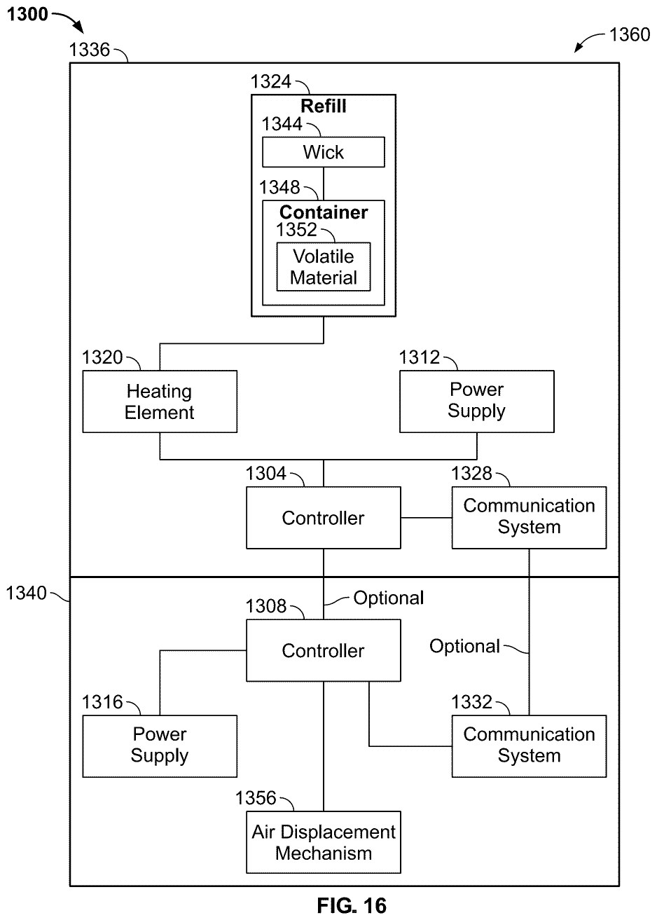

Referring now to FIGS. 15 and 16, another volatile material dispenser 1300 is shown. The volatile material dispenser 1300 of FIGS. 15 and 16 is similar to the volatile material dispenser 100 of FIG. 1, but includes differences as outlined below. The volatile material dispenser 1300 comprises a first controller 1304 and a second controller 1308. The first controller 1304 is in communication with a first power supply 1312 and a second controller 1308 is in communication with a second power supply 1316. The first controller 1304 is in communication with a heating element 1320 that is in communication with a refill 1324. It will be appreciated that the volatile material dispenser 1300 of FIG. 15 and FIG. 16 include components that are similar (structurally and/or functionally similar) with components of the volatile material dispenser 100 of FIG. 1. For example, the first controller 1304 and the second controller 1308 of FIGS. 15 and 16 may be similar to the controller 104 of FIG. 1. Additionally, the heating element 1320 may be similar to the heating element 120 of FIG. 1. The first power supply 1312 and the second power supply 1316 and the refill 1324 may be similar to the power supply 108 and the refill 124 of FIG. 1. First and second communication systems 1328, 1332 may be similar to the communication system 420 of FIG. 6. The first power supply 1312 and the second power supply 1316 may be a unitary power supply or separate power supplies. In some embodiments, the first power supply 1312 is disposed within a housing 1336 and is a battery that can be recharged when in electrical communication with the second power supply 1316 that is disposed within a base 1340. The second power supply 1316 may be a wall outlet or a USB-C type plug, that can be used to charge or power the first power supply 1312. In some embodiments, there is only a first power supply 1312 that is configured to store and receive power.

Referring to FIG. 15, the volatile material dispenser 1300 is shown in a first configuration 1342 wherein the housing 1336 is separated from the base 1340. The housing 1336 of the volatile material dispenser 1300 contains the refill 1324 that includes a wick 1344 and a container 1348 that has a volatile material 1352 therein. The housing 1336 may also contain the first controller 1304 that is in communication with the first power supply 1312, the heating element 1320, and the first communication system 1328. In some embodiments, the first and second communication systems 1328, 1332 may be an integral part of the first and second controller 1304, 1308 respectively. The housing 1336 may be configured to have a stand, such as legs, which are configured to elevate the housing 1336 above the surface upon which it is placed. The elevation of the housing 1336 may allow for air to flow through upward, between the legs and into the housing 1336 to facilitate the dispersion of the evaporated volatile material 1352 into the surrounding environment.

Still referring to FIG. 15, a base 1340 of the volatile material dispenser 1300 is configured to be detachably coupled to the housing 1336. The base 1340 may contain the second controller 1308 connected to the second communication system 1332, the second power supply 1316, and an air displacement mechanism 1356. Since the housing 1336 and the base 1340 are configured to be detachably coupled, the housing 1336 can be moved and operated within an environment and placed in locations that may not be feasible to be placed when the volatile material dispenser 1300 is coupled to a wall outlet. For example, the housing 1336 could be placed and operated in the middle of a table that is not located near an outlet. After one or more operational cycles, the housing 1336 may be removed from the table and reconnected back to the base 1340 to recharge the first power supply 1312 using the second power supply 1316.

Referring now to FIG. 16, a second configuration 1360 of the volatile material dispenser 1300 is shown. In FIG. 16, the housing 1336 and the base 1340 are coupled together. In some embodiments, the coupling may be achieved by one or more magnets, a lock and key configuration, a clip, and/or any other fastening mechanism. When the base 1340 and the housing 1336 are coupled together as shown in FIG. 16, the volatile material dispenser 1300 may be configured to register using a sensor (not shown) that the housing 1336 and the base 1340 are coupled together. The information that the housing 1336 and the base 1340 are coupled together may be communicated to the first controller 1304 and/or the second controller 1308. If the first controller 1304 and/or the second controller 1308 receives a signal that the base 1340 and the housing 1336 are coupled, there may be a different operational cycle that one or both of the controllers 1304, 1308 are configured to initiate. For example, the first controller 1304 may output a larger power output to the heating element 1320 if the base 1340 and the housing 1336 are coupled to one another. By outputting a larger output power, more power is used and more volatile material 1352 may be evaporated and dispersed into the surrounding environment.

The increase in evaporated volatile material 1352 may be accomplished without saturating the surrounding environment with evaporated volatile material 1352 by utilizing the air displacement mechanism 1356. The air displacement mechanism 1356 is configured to facilitate the dispersion of the evaporated volatile material 1352 into the surrounding environment. Further, the additional power used by the heating element 1320 may be sustained by using a combination of one or both power supplies 1312, 1316 that may not be feasible if they were not connected. In some embodiments, the volatile material dispenser 1300 may be configured to output less power to the heating element 1320 when the housing 1336 and the base 1340 are not coupled. When the housing 1336 is not coupled with the base 1340, the input power to the heating element 1320 may be reduced when compared to when the housing 1336 and the base 1340 are connected. If the housing 1336 is not connected to the base 1340, the first controller 1304 may output less power to the heating element 1320 to prevent a high concentration of evaporated volatile material 1352 from building up in and near the volatile material dispenser 1300. The volatile material dispenser 1300 may use the communication systems 1328, 1332 to send and receive signals to allow the controllers 1304, 1308 to know whether the housing 1336 and the base 1340 are connected.

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein.

Any of the embodiments described herein may be modified to include any of the structures or methodologies disclosed in connection with different embodiments. Further, the present disclosure is not limited to the dispensing systems of the type specifically shown. Still further, the methods and systems of any of the embodiments disclosed herein may be modified to work with any type of volatile material dispenser.

### INDUSTRIAL APPLICABILITY

Numerous modifications to the present disclosure will be apparent to those skilled in the art in view of the foregoing description. Accordingly, this description is to be construed as illustrative only and is presented for the purpose of enabling those skilled in the art to make and use the disclosure. The exclusive rights to all modifications which come within the scope of the appended claims are reserved.

1. A volatile material dispenser, comprising:
a power supply;
a heating element;
a first temperature detection device;
a second temperature detection device;
a refill; and
a controller,
wherein the controller is in communication with the power supply, the heating element, the first temperature detection device, and the second temperature detection device,
wherein the first temperature detection device is in thermal communication with the refill; and
wherein the heating element is in thermal communication with the refill and the second temperature detection device.

2. The volatile material dispenser of claim 1, wherein the first temperature detection device or the second temperature detection device is a thermistor.

3. The volatile material dispenser of claim 1, wherein the first temperature detection device or the second temperature detection device is a thermocouple.

4. The volatile material dispenser of claim 1, wherein the volatile material dispenser also comprises an air displacement mechanism.

5. The volatile material dispenser of claim 4, wherein the air displacement mechanism is an air vent.

6. The volatile material dispenser of claim 1, wherein the volatile material dispenser also comprises an air flow detection mechanism that is in communication with the controller.

7. The volatile material dispenser of claim 1, wherein the volatile material dispenser further comprises a volatile material concentration detector that is in communication with the controller.

8. The volatile material dispenser of claim 1, wherein the volatile material dispenser is in communication with one or more smart home devices via a communication network.

9.-29. (canceled)

30. A volatile material dispenser, comprising:
a power supply;
a heating element;
a temperature detection device;
a refill; and
a controller,
wherein the controller is in communication with the power supply, the heating element, and the temperature detection device,
wherein the temperature detection device is in thermal communication with the refill, and
wherein the heating element is in thermal communication with the refill.

31. The volatile material dispenser of claim 30, wherein the refill comprises a cartridge, a volatile material, and a wick.

32. The volatile material dispenser of claim 31, wherein the temperature detection device is in thermal communication with the wick.

33. (canceled)

34. (canceled)

35. The volatile material dispenser of claim 30, wherein the volatile material dispenser also comprises an air displacement mechanism, and
wherein the air displacement mechanism is an air vent.

36. (canceled)

37. The volatile material dispenser of claim 30, wherein the volatile material dispenser further comprises an air flow detection mechanism that is in communication with the controller.

38. The volatile material dispenser of claim 30, wherein the volatile material dispenser further comprises a volatile material concentration detector that is in communication with the controller.

39. (canceled)

40. A volatile material dispenser, comprising:
a housing that comprises a first controller that is in electrical communication with a heating element that is in thermal communication with a refill and electrical communication with a first power supply; and
a base that comprises a second controller that is electrical communication with an air displacement mechanism and a second power supply,
wherein the housing and the base can be configured to be removably coupled with each other.

41. The volatile material dispenser of claim 40, wherein the volatile material dispenser has at least one operational cycle when the base is not coupled to the housing.

42. The volatile material dispenser of claim 41, wherein the volatile material dispenser has at least one communication system that is connected to at least one controller.

43. The volatile material dispenser of claim 41, wherein the volatile material dispenser has at least one operational cycle when the base is coupled to the housing that is different than at least one operational cycle when the base is not coupled to the housing.

44. The volatile material dispenser of claim 43, wherein the volatile material dispenser has a first communication system and a second communication system,
wherein the first communication system is connected to the first controller, and
wherein the second communication system is connected to the second controller.

45. (canceled)

46. The volatile material dispenser of claim 40, wherein the refill further comprises a wick and a container containing a volatile material within.
